SEQUENCE LISTING (17 sequences)

SEQ ID NO: 1 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 2 - protein, 239 aa, Artificial Sequence
  Description of Artificial Sequence: Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 3 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 4 - protein, 239 aa, Artificial Sequence
  Description of Artificial Sequence: Synthetic polypeptide
MVSKGEELFTGVVPILVELDGDVNGHKFSVSGEGEGDATYGKLTLKFVLTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 5 - protein, 239 aa, Artificial Sequence
  Description of Artificial Sequence: Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYNASLSKDPNEKRDHMVLLEFVTAAGITLGMDELYK

SEQ ID NO: 6 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKVNFKIRHNIEDGGVQLADHYQQNTPIGDGPVLLPDNHYLSYQSKLSKDPNEKRDHMVLLEFVTAAGITLGMDELYK

SEQ ID NO: 7 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHMKQHDFFKSAMPEGYVQERTIFFKDDGNYKTRAEVKFEGDTLVNRIELKGIDFKEDGNILGHKLEYNYNCHNVYITADKQKNGIKANFKIRHNIEDGGVQLADHYQQNTPIGDGPVLLPDNHYLSYQSKLSKDPNEKRDHMVLLEFVTAAGITLSMDELYK

SEQ ID NO: 8 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHMKQHDFFKSAMPEGYVQERTIFFKDDGNYKTRAEVKFEGDTLVNRIELKGIDFKEDGNILGHKLEYNYNNHNVYITADRQKNGIKANFKIRHNIEDGGVQLADHYQQNTPIGDGPVLLPDNHYLSYQSKLSKDPNEKRDHMVLLEFVTAAGITLGMDELYK

SEQ ID NO: 9 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHLKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 10 - protein, 239 aa, Artificial Sequence
  Description of Artificial Sequence: Synthetic polypeptide
MVSKGEELFTGVVPILVELDGDVNGHKFSVSGEGEGDATYGKLTLKFICTTGKLPVPWPTLVTSLGYGLQCFARYPDHMKQHDFFKSAMPEGYVQERTIFFKDDGNYKTRAEVKFEGDTLVNRIELKGIDFKEDGNILGHKLEYNYNSHNVCITADKQKNGIKANFKIRHNIEDGGVQLADHYQQNTPIGDGPVLLPDNHYLSYQSKLSKDPNEKRDHMVLLEFVTAAGITLGMDELYK

SEQ ID NO: 11 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 12 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacgggcacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggtcaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 13 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacatgaagcggcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaactgccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcagcatggacgagctgtacaag

SEQ ID NO: 14 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacaaccacaacgtctatatcaccgccgacaggcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 15 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacttgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 16 - DNA, 717 bp, Artificial Sequence
  Description of Artificial Sequence: Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccagcctgggctacggcctgcagtgcttcgcccgctaccccgaccacatgaagcagcacgacttcttcaagtcagccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctgtatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaag

SEQ ID NO: 17 - protein, 6 aa, Artificial Sequence
  Description of Artificial Sequence: Synthetic 6xHis tag
HHHHHH

(19) United States
(12) Patent Application Publication
(10) Pub. No.: US 2023/0203110 A1
(43) Pub. Date: Jun. 29, 2023

(54) PHOTOSTABLE FLUORESCENT PROTEINS

(71) Applicants: Baylor College of Medicine, Houston, TX (US); William Marsh Rice University, Houston, TX (US)

(72) Inventors: François St-Pierre, Houston, TX (US); Jihwan Lee, Houston, TX (US); Zhuohe Liu, Houston, TX (US)

(21) Appl. No.: 17/759,323

(22) PCT Filed: Jan. 25, 2021
(86) PCT No.: PCT/US2021/014927

§ 371 Date: Jul. 22, 2022

Related U.S. Application Data
(60) Provisional application No. 62/965,274, filed on Jan. 24, 2020.

(51) Int. Cl. C07K 14/435; G01N 33/533; G01N 33/58

(52) U.S. Cl. CPC C07K 14/43595; G01N 33/533; G01N 33/582; C07K 2319/60

(57) ABSTRACT

Provided herein are photostable fluorescent proteins and methods of making and using those proteins. A photostable fluorescent protein variant is provided comprising an amino acid sequence that is at least 90% identical to SEQ ID NO:3, wherein the amino acid sequence comprises a first substitution at a residue corresponding to residue 46 of a polypeptide of SEQ ID NO:2 and a second substitution at a residue corresponding to residue 63 of the polypeptide of SEQ ID NO:2, and the variant is more photostable than the polypeptide of SEQ ID NO:2. A method of detecting a protein of interest is also provided, comprising the steps of expressing a fusion protein and detecting the fluorescence of the fusion protein, thereby detecting the protein of interest.

### CROSS-REFERENCE TO RELATED APPLICATIONS

This application claims priority to U.S. Provisional Pat. Application Serial No. 62/965,274, filed Jan. 24, 2020, which is incorporated by reference herein in its entirety.

### STATEMENT REGARDING FEDERALLY SPONSORED RESEARCH OR DEVELOPMENT

This invention was made with government support under EB027145 and NS 113294 awarded by the National Institutes of Health, and 1707359 and 1935265 awarded by the National Science Foundation. The government has certain rights in the invention.

### TECHNICAL FIELD

Embodiments of the disclosure relate generally and at least to the fields of biochemistry, cell biology, molecular biology, and medical diagnostics. More particularly, the disclosure is directed to the use and production of fluorescent proteins.

### BACKGROUND

In biochemistry, molecular biology and medical diagnostics, it is often desirable to add a fluorescent label to a protein so that the protein can be easily tracked and quantified. Fluorescent proteins have become commonly used reporter molecules for examining various cellular processes, including the regulation of gene expression, the localization and interactions of cellular proteins, the pH of intracellular compartments, and the activities of enzymes.

However, all organic fluorophores undergo irreversible photobleaching, losing brightness with prolonged illumination. Some molecules undergo reversible photobleaching. While fluorescent proteins typically bleach at a substantially slower rate than many small molecule dyes, a lack of photostability remains an important limiting factor for experiments requiring large numbers of images of single cells. Screening methods focusing solely on brightness or wavelength can be highly effective in optimizing both properties, but the absence of selective pressure for photostability in such screens leads to unpredictable photobleaching behavior in the resulting fluorescent proteins.

Yellow fluorescent proteins (YFPs) are one type of fluorescent protein commonly used in vitro and in vivo as fusion proteins, gene expression reporters, and protein biosensors. However, like all fluorescent proteins, YFPs also suffer from photobleaching, thereby hindering their deployment in applications requiring long-term imaging of cellular activity or high signal stability. Therefore, there is a strong desire to develop more photostable variants of YFPs.

### BRIEF SUMMARY

Embodiments of the present disclosure are directed to compositions of fluorescent protein variants, including yellow, green, blue, or red (for example) fluorescent protein variants, including those that show significantly improved photostability as compared to commonly used fluorescent proteins. Specific embodiments of the disclosure are directed to compositions of yellow fluorescent protein variants, including those that show significantly improved photostability as compared to commonly used yellow fluorescent protein (YFP). The variants of the present disclosure are based on derivatives of mVenus, a YFP with high brightness, low acid sensitivity, and wide usage as an individual tag and within biosensors.

Some embodiments of the disclosure encompass yellow fluorescent protein variants with up to a 4-fold improvement in photostability with no significant change in brightness. In at least some cases, the variants are more photostable and just as bright as a precursor including one in bacterial, yeast, and mammalian cells, and with widefield illumination or laser scanning confocal illumination. The identification of these variants facilitates studies in which photostability is important, such as when conducting time lapse imaging to follow cellular activity over prolonged durations or when high light power is necessary for detection of a small number of molecules, for example.

Embodiments of the disclosure encompass a photostable fluorescent protein variant comprising an amino acid sequence that is at least 90% (or 91%, or 92%, or 93%, or 94%) identical to SEQ ID NO:3, wherein the amino acid sequence comprises a substitution at a residue corresponding to residue 46 of a polypeptide of SEQ ID NO:2, and the variant is more photostable than the polypeptide of SEQ ID NO:2. In specific cases, the substitution is an L46F substitution. In some cases, the photostable fluorescent protein variant has an amino acid sequence that is at least 95%, 96%, 97%, 98%, or 99% identical to SEQ ID NO:3.

Embodiments of the disclosure include photostable fluorescent protein variants comprising an amino acid sequence that is at least 90% (or 91%, or 92%, or 93%, or 94%) identical to SEQ ID NO:1, wherein the amino acid sequence comprises a first substitution at a residue corresponding to residue 46 of a polypeptide of SEQ ID NO:2 and a second substitution at a residue corresponding to residue 63 of the polypeptide of SEQ ID NO:2, and the variant is more photostable than the polypeptide of SEQ ID NO:2. In specific cases, the first substitution is an L46F substitution and the second substitution is a T63S substitution. In specific embodiments, the amino acid sequence is at least 95%, 96%, 97%, 98%, or 99% identical to SEQ ID NO:1. In specific cases, the amino acid sequence is identical to SEQ ID NO:1.

Embodiments of the disclosure include photostable fluorescent protein variants comprising an amino acid sequence that is at least 90% (or 91%, or 92%, or 93%, or 94%) identical to SEQ ID NO:4, wherein the amino acid sequence comprises a first substitution at a residue corresponding to residue 46 of a polypeptide of SEQ ID NO:2, a second substitution at a residue corresponding to residue 47 of the polypeptide of SEQ ID NO:2, and a third substitution at a residue corresponding to residue 48 of the polypeptide of SEQ ID NO:2, and the variant is more photostable than the polypeptide of SEQ ID NO:2. In specific cases, the first substitution is an L46F substitution, the second substitution is an I47V substitution, and the third substitution is a C48L substitution. In particular embodiments, the amino acid sequence is at least 95%, 96%, 97%, 98%, or 99% identical to SEQ ID NO:4. In specific aspects, the photostable fluorescent protein variant has an amino acid sequence that is identical to SEQ ID NO:4.

Embodiments of the disclosure include photostable fluorescent protein variants comprising an amino acid sequence that is at least 90% (or 91%, or 92%, or 93%, or 94%) identical to SEQ ID NO:5, wherein the amino acid sequence comprises a first substitution at a residue corresponding to residue 204 of a polypeptide of SEQ ID NO:2, a second substitution at a residue corresponding to residue 205 of the polypeptide of SEQ ID NO:2, and a third substitution at a residue corresponding to residue 206 of the polypeptide of SEQ ID NO:2, and the variant is the same as or more photostable than the polypeptide of SEQ ID NO:2. In certain aspects, the first substitution is a Q204N substitution, the second substitution is an S205A substitution, and the third substitution is a K206S substitution. In specific embodiments, the amino acid sequence is at least 95%, 96%, 97%, 98%, or 99% identical to SEQ ID NO:5. The amino acid sequence may be identical to SEQ ID NO:5.

Embodiments of the disclosure include photostable fluorescent protein variants comprising an amino acid sequence that is at least 90% (or 91%, or 92%, or 93%, or 94%) identical to SEQ ID NO:6, wherein the amino acid sequence comprises a first substitution at a residue corresponding to residue 46 of a polypeptide of SEQ ID NO:2, a second substitution at a residue corresponding to residue 63 of the polypeptide of SEQ ID NO:2, and a third substitution at a residue corresponding to residue 163 of the polypeptide of SEQ ID NO:2, and the variant is more photostable than the polypeptide of SEQ ID NO:2. In specific cases, the first substitution is an L46F substitution, the second substitution is a T63S substitution, and the third substitution is an A163V substitution. In specific embodiments, the amino acid sequence is at least 95%, 96%, 97%, 98%, or 99% identical to SEQ ID NO:6. In specific cases, the amino acid sequence is identical to SEQ ID NO:6.

Embodiments of the disclosure include photostable fluorescent protein variants comprising an amino acid sequence that is at least 90% (or 91%, or 92%, or 93%, or 94%) identical to SEQ ID NO:7, wherein the amino acid sequence comprises a first substitution at a residue corresponding to residue 46 of a polypeptide of SEQ ID NO:2, a second substitution at a residue corresponding to residue 63 of the polypeptide of SEQ ID NO:2, a third substitution at a residue corresponding to residue 80 of the polypeptide of SEQ ID NO:2, a fourth substitution at a residue corresponding to 147 of the polypeptide of SEQ ID NO:2, and a fifth substitution at a residue corresponding to 232 of the polypeptide of SEQ ID NO:2, and the variant is more photostable than the polypeptide of SEQ ID NO:2. In specific cases, the first substitution is an L46F substitution, the second substitution is a T63S substitution, the third substitution is a Q80R substitution, the fourth substitution is an S 147C substitution, and the fifth substitution is a G232S substitution. In specific embodiments, the amino acid sequence is at least 95%, 96%, 97%, 98%, or 99% identical to SEQ ID NO:7. In specific cases, the amino acid sequence is identical to SEQ ID NO:7.

Embodiments of the disclosure include photostable fluorescent protein variants comprising an amino acid sequence that is at least 90% (or 91%, or 92%, or 93%, or 94%) identical to SEQ ID NO:8, wherein the amino acid sequence comprises a first substitution at a residue corresponding to residue 46 of a polypeptide of SEQ ID NO:2, a second substitution at a residue corresponding to residue 63 of the polypeptide of SEQ ID NO:2, a third substitution at a residue corresponding to residue 147 of the polypeptide of SEQ ID NO:2, and a fourth substitution at a residue corresponding to residue 156 of the polypeptide of SEQ ID NO:2, and the variant is more photostable than the polypeptide of SEQ ID NO:2. In specific cases, the first substitution is an L46F substitution, the second substitution is a T63S substitution, the third substitution is an S147N substitution, and the fourth substitution is a K156R substitution. In specific embodiments, the amino acid sequence is at least 95%, 96%, 97%, 98%, or 99% identical to SEQ ID NO:8. In specific cases, the amino acid sequence is identical to SEQ ID NO:8.

Embodiments of the disclosure include photostable fluorescent protein variants comprising an amino acid sequence that is at least 90% (or 91%, or 92%, or 93%, or 94%) identical to SEQ ID NO:9, wherein the amino acid sequence comprises a first substitution at a residue corresponding to residue 46 of a polypeptide of SEQ ID NO:2, a second substitution at a residue corresponding to residue 63 of the polypeptide of SEQ ID NO:2, and a third substitution at a residue corresponding to residue 78 of the polypeptide of SEQ ID NO:2, and the variant is more photostable than the polypeptide of SEQ ID NO:2. In specific cases, the first substitution is an L46F substitution, the second substitution is a T63S substitution, and the third substitution is an M78L substitution. In specific embodiments, the amino acid sequence is at least 95%, 96%, 97%, 98%, or 99% identical to SEQ ID NO:9. In specific cases, the amino acid sequence is identical to SEQ ID NO:9.

Embodiments of the disclosure include photostable fluorescent protein variants comprising an amino acid sequence that is at least 90% (or 91%, or 92%, or 93%, or 94%) identical to SEQ ID NO:10, wherein the amino acid sequence comprises a first substitution at a residue corresponding to residue 46 of a polypeptide of SEQ ID NO:2, a second substitution at a residue corresponding to residue 63 of the polypeptide of SEQ ID NO:2, and a third substitution at a residue corresponding to residue 151 of the polypeptide of SEQ ID NO:2, and the variant is more photostable than the polypeptide of SEQ ID NO:2. In specific cases, the first substitution is an L46F substitution, the second substitution is a T63S substitution, and the third substitution is an Y151C substitution. In specific embodiments, the amino acid sequence is at least 95%, 96%, 97%, 98%, or 99% identical to SEQ ID NO:10. In specific cases, the amino acid sequence is identical to SEQ ID NO:10.

In particular embodiments, the disclosure encompasses fusion proteins comprising any photostable fluorescent protein variant of the disclosure operatively linked to a protein of interest. In some embodiments, there is provided a tandem fluorescent protein comprising a first photostable fluorescent protein variant operatively linked to a second fluorescent protein.

Certain embodiments include nucleic acid molecules comprising a nucleic acid sequence encoding a photostable fluorescent protein variant of the disclosure. In specific embodiments, the nucleic acid sequence is operably linked to a promoter region of interest. The nucleic acid sequence may be expressed when in a cell. The nucleic acid sequence may encode a fusion protein comprising the photostable fluorescent protein variant. In some cases, the nucleic acid sequence is identical to SEQ ID NO:11, SEQ ID NO:12, SEQ ID NO:13, SEQ ID NO:14, or SEQ ID NO:15 or is at least 75%, 80%, 85%, 90%, 91%, 92%, 93%, 94%, 95%, 96%, 97%, 98%, or 99% identical to SEQ ID NO:11, SEQ ID NO:12, SEQ ID NO:13, SEQ ID NO:14, or SEQ ID NO:15. Any nucleic acid of the disclosure may be comprised in a vector of any kind (including viral (retroviral, lentiviral, adenoviral, or adeno-associated) or non-viral). In specific embodiments, there is an expression cassette comprising: a transcriptional initiation region that is functional in an expression host; any nucleic acid molecule of the disclosure; and a transcriptional termination region functional in the expression host. In specific embodiments, there is a host cell, or plurality or progeny thereof, comprising the expression cassette encompassed herein as part of an extrachromosomal element or integrated into the genome of a host cell as a result of introduction of the expression cassette into the host cell. The cells may be transgenic cells, or progeny thereof, comprising the nucleic acid molecule of the disclosure.

In particular embodiments, there is a method of detecting a protein of interest, comprising the steps of: expressing a fusion protein of the disclosure; and detecting the fluorescence of the fusion protein.

In certain embodiments, there is a method of detecting the subcellular localization of a protein of interest, comprising the steps of: expressing in a cell a fusion protein of the disclosure; detecting the fluorescence of the fusion protein; and determining the subcellular location of the fluorescence within the cell.

In specific embodiments, there is a method of detecting the motility of a protein of interest, comprising the steps of: expressing in a cell a fusion protein of the disclosure; performing time-sequential observations of fluorescence in the cell; and detecting differences in the fluorescence between the time-sequential observations.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A-1B are a chart and graphic, respectively, illustrating the residues selected for mutagenesis;

FIG. 2 is an overview diagram showing the screening platform applied to engineered mutagenesis libraries;

FIGS. 3A-3C are charts comparing the photostability of mGold and wild-type mVenus in yeast and mammalian cells photobleached with widefield illumination as well as mammalian cells photobleached with laser scanning confocal illumination;

FIGS. 4A-4B are charts comparing, in yeast, the photostability and brightness of four photostable yellow fluorescent protein variants to three commonly used yellow fluorescent proteins;

FIGS. 5A-5B are charts comparing the photostability and brightness of mGold to three commonly used yellow fluorescent proteins in yeast and human cells;

FIGS. 6A-6B are charts of the photostability at different irradiances in yeast and mammalian cells;

FIGS. 7A-7H are images of mGold subcellular localization in individual HeLa cells and FIGS. 7I-7P are zoomed out images of multiple cells expressing the mGold constructs;

FIG. 8 shows the normalized cytotoxicity profile of mGold as compared to wild-type mVenus;

FIG. 9 shows the in vitro excitation and emission spectra of mGold as compared to wild-type mVenus; and

FIG. 10 shows the elution profile of mGold as compared to tdTomato, mCherry, and wild-type mVenus.

### DETAILED DESCRIPTION

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the disclosure. For purposes of the present disclosure, the following terms are defined.

The term “polypeptide” or “protein” refers to a polymer of two or more amino acid residues. The terms apply to amino acid polymers in which one or more amino acid residue is an artificial chemical analogue of a corresponding naturally occurring amino acid, as well as to naturally occurring amino acid polymers. The term “recombinant protein” refers to a protein that is produced by expression of a nucleotide sequence encoding the amino acid sequence of the protein from a recombinant DNA molecule.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The following sequences are referred to herein as follows, and the numbering for the mutations (e.g., L46F) is with reference to the initial methionine in the sequence to be amino acid residue 0. Correspondingly, the first valine of each listed sequence is amino acid residue 1:

mGold (mVenus(L46F, T63S)) (SEQ ID NO:1)
M VSKGEELFTG VVPILVELDG DVNGHKFSVS GEGEGDATYG KLTLKFICTT
GKLPVPWPTL VTSLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYQSKLSKD PNEKRDHMVL LEFVTAAGIT LGMDELYK

Wildtype mVenus (SEQ ID NO:2)
M VSKGEELFTG VVPILVELDG DVNGHKFSVS GEGEGDATYG KLTLKLICTT
GKLPVPWPTL VTTLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYQSKLSKD PNEKRDHMVL LEFVTAAGIT LGMDELYK

mVenus(L46F) (SEQ ID NO:3)
M VSKGEELFTG VVPILVELDG DVNGHKFSVS GEGEGDATYG KLTLKFICTT
GKLPVPWPTL VTTLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYQSKLSKD PNEKRDHMVL LEFVTAAGIT LGMDELYK

mVenus(L46F, I47V, C48L) (SEQ ID NO:4)
M VSKGEELFTG VVPILVELDG DVNGHKFSVS GEGEGDATYG KLTLKFVLTT
GKLPVPWPTL VTTLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYQSKLSKD PNEKRDHMVL LEFVTAAGIT LGMDELYK

mVenus(Q204N, S205A, K206S) (SEQ ID NO:5)
M VSKGEELFTG VVPILVELDG DVNGHKFSVS GEGEGDATYG KLTLKLICTT
GKLPVPWPTL VTTLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYNASLSKD PNEKRDHMVL LEFVTAAGIT LGMDELYK

mVenus(L46F, T63S, A163V) (SEQ ID NO:6)
M VSKGEELFTG VVPILVELDG DVNGHKFSVS GEGEGDATYG KLTLKFICTT
GKLPVPWPTL VTSLGYGLQC FARYPDHMKQ HDFFKSAMPE GYVQERTIFF
KDDGNYKTRA EVKFEGDTLV NRIELKGIDF KEDGNILGHK LEYNYNSHNV
YITADKQKNG IKVNFKIRHN IEDGGVQLAD HYQQNTPIGD GPVLLPDNHY
LSYQSKLSKD PNEKRDHMVL LEFVTAAGIT LGMDELYK

mVenus(L46F, T63S, Q80R, S147C, G232S) (SEQ ID NO:7)
M VSKGEELFTG VVPILVELDG DVNGHKFSVS GEGEGDATYG KLTLKFICTT
GKLPVPWPTL VTSLGYGLQC FARYPDHMKQ HDFFKSAMPE GYVQERTIFF
KDDGNYKTRA EVKFEGDTLV NRIELKGIDF KEDGNILGHK LEYNYNCHNV
YITADKQKNG IKANFKIRHN IEDGGVQLAD HYQQNTPIGD GPVLLPDNHY
LSYQSKLSKD PNEKRDHMVL LEFVTAAGIT LSMDELYK

mVenus(L46F, T63S, S147N, K156R) (SEQ ID NO:8)
M VSKGEELFTG VVPILVELDG DVNGHKFSVS GEGEGDATYG KLTLKFICTT
GKLPVPWPTL VTSLGYGLQC FARYPDHMKQ HDFFKSAMPE GYVQERTIFF
KDDGNYKTRA EVKFEGDTLV NRIELKGIDF KEDGNILGHK LEYNYNNHNV
YITADRQKNG IKANFKIRHN IEDGGVQLAD HYQQNTPIGD GPVLLPDNHY
LSYQSKLSKD PNEKRDHMVL LEFVTAAGIT LGMDELYK

mVenus(L46F, T63S, M78L) (SEQ ID NO:9)
M VSKGEELFTG VVPILVELDG DVNGHKFSVS GEGEGDATYG KLTLKFICTT
GKLPVPWPTL VTSLGYGLQC FARYPDHLKQ HDFFKSAMPE GYVQERTIFF
KDDGNYKTRA EVKFEGDTLV NRIELKGIDF KEDGNILGHK LEYNYNSHNV
YITADKQKNG IKANFKIRHN IEDGGVQLAD HYQQNTPIGD GPVLLPDNHY
LSYQSKLSKD PNEKRDHMVL LEFVTAAGIT LGMDELYK

mVenus(L46F, T63S, Y151C) (SEQ ID NO:10)
M VSKGEELFTG VVPILVELDG DVNGHKFSVS GEGEGDATYG KLTLKFICTT
GKLPVPWPTL VTSLGYGLQC FARYPDHMKQ HDFFKSAMPE GYVQERTIFF
KDDGNYKTRA EVKFEGDTLV NRIELKGIDF KEDGNILGHK LEYNYNSHNV
CITADKQKNG IKANFKIRHN IEDGGVQLAD HYQQNTPIGD GPVLLPDNHY
LSYQSKLSKD PNEKRDHMVL LEFVTAAGIT LGMDELYK

mGold (SEQ ID NO:11)
ATGGTGAGCA AGGGCGAGGA GCTGTTCACC GGGGTGGTGC CCATCCTGGT
CGAGCTGGAC GGCGACGTAA ACGGCCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACATGAAG CAGCACGACT
TCTTCAAGTC CGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CAGCCACAAC
GTCTATATCA CCGCCGACAA GCAGAAGAAC GGCATCAAGG CCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCGGCA
TGGACGAGCT GTACAAG

mVenus(L46F, T63S, A163V) (SEQ ID NO:12)
ATGGTGAGCA AGGGCGAGGA GCTGTTCACC GGGGTGGTGC CCATCCTGGT
CGAGCTGGAC GGCGACGTAA ACGGGCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACATGAAG CAGCACGACT
TCTTCAAGTC CGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CAGCCACAAC
GTCTATATCA CCGCCGACAA GCAGAAGAAC GGCATCAAGG TCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCGGCA
TGGACGAGCT GTACAAG

mVenus(L46F, T63S, Q80R, S147C, G232S) (SEQ ID NO:13)
ATGGTGAGCA AGGGCGAGGA GCTGTTCACC GGGGTGGTGC CCATCCTGGT
CGAGCTGGAC GGCGACGTAA ACGGCCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACATGAAG CGGCACGACT
TCTTCAAGTC CGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CTGCCACAAC
GTCTATATCA CCGCCGACAA GCAGAAGAAC GGCATCAAGG CCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCAGCA
TGGACGAGCT GTACAAG

mVenus(L46F, T63S, S147N, K156R) (SEQ ID NO:14)
ATGGTGAGCA AGGGCGAGGA GCTGTTCACC GGGGTGGTGC CCATCCTGGT
CGAGCTGGAC GGCGACGTAA ACGGCCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACATGAAG CAGCACGACT
TCTTCAAGTC CGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CAACCACAAC
GTCTATATCA CCGCCGACAG GCAGAAGAAC GGCATCAAGG CCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCGGCA
TGGACGAGCT GTACAAG

mVenus(L46F, T63S, M78L) (SEQ ID NO:15)
ATGGTGAGCA AGGGCGAGGA GCTGTTCACC GGGGTGGTGC CCATCCTGGT
CGAGCTGGAC GGCGACGTAA ACGGCCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACTTGAAG CAGCACGACT
TCTTCAAGTC CGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CAGCCACAAC
GTCTATATCA CCGCCGACAA GCAGAAGAAC GGCATCAAGG CCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCGGCA
TGGACGAGCT GTACAAG

mVenus(L46F, T63S, Y151C) (SEQ ID NO:16)
ATGGTGAGCA AGGGCGAGGA GCTGTTCACC GGGGTGGTGC CCATCCTGGT
CGAGCTGGAC GGCGACGTAA ACGGCCACAA GTTCAGCGTG TCCGGCGAGG
GCGAGGGCGA TGCCACCTAC GGCAAGCTGA CCCTGAAGTT CATCTGCACC
ACCGGCAAGC TGCCCGTGCC CTGGCCCACC CTCGTGACCA GCCTGGGCTA
CGGCCTGCAG TGCTTCGCCC GCTACCCCGA CCACATGAAG CAGCACGACT
TCTTCAAGTC AGCCATGCCC GAAGGCTACG TCCAGGAGCG CACCATCTTC
TTCAAGGACG ACGGCAACTA CAAGACCCGC GCCGAGGTGA AGTTCGAGGG
CGACACCCTG GTGAACCGCA TCGAGCTGAA GGGCATCGAC TTCAAGGAGG
ACGGCAACAT CCTGGGGCAC AAGCTGGAGT ACAACTACAA CAGCCACAAC
GTCTGTATCA CCGCCGACAA GCAGAAGAAC GGCATCAAGG CCAACTTCAA
GATCCGCCAC AACATCGAGG ACGGCGGCGT GCAGCTCGCC GACCACTACC
AGCAGAACAC CCCCATCGGC GACGGCCCCG TGCTGCTGCC CGACAACCAC
TACCTGAGCT ACCAGTCCAA GCTGAGCAAA GACCCCAACG AGAAGCGCGA
TCACATGGTC CTGCTGGAGT TCGTGACCGC CGCCGGGATC ACTCTCGGCA
TGGACGAGCT GTACAAG

In certain embodiments, the present disclosure concerns novel compositions comprising at least one proteinaceous molecule. As used herein, a “proteinaceous molecule,” “proteinaceous composition,” “proteinaceous compound,” “proteinaceous chain” or “proteinaceous material” generally refers, but is not limited to, a protein of greater than about 50, 75, 100, 125, 150, 175, 200, 210, 220, 225, 230, 235, or more amino acids or the full length endogenous sequence translated from a gene; a polypeptide of greater than about 50, 75, 100, 125, 150, 175, 200, 210, 220, 225, 230, 235, or more amino acids; and/or a peptide of from about 3 to about 100 amino acids. All the “proteinaceous” terms described above may be used interchangeably herein, including the term “protein”.

In certain embodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 231, about 232, about 233, about 234, about 235, about 236, about 237, about 238, about 239, or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

In particular embodiments, the protein of the disclosure comprises various combinations of the following amino acids:
alanine - ala - A;
arginine - arg - R;
asparagine - asn - N;
aspartic acid - asp - D;
cysteine - cys - C;
glutamine - gln - Q;
glutamic acid - glu - E;
glycine - gly - G;
histidine - his - H;
isoleucine - ile - I;
leucine - leu - L;
lysine - lys - K;
methionine - met - M;
phenylalanine - phe - F;
proline - pro - P;
serine - ser - S;
threonine - thr - T;
tryptophan - trp - W;
tyrosine - tyr - Y; and
valine - val - V.

However, in some embodiments, the term “proteinaceous composition” or “protein” encompasses amino molecule sequences comprising at least one of the 20 common amino acids in naturally synthesized proteins in addition to at least one modified or unusual amino acid, including but not limited to those shown on Table 1 below.

TABLE 1

Modified and Unusual Amino Acids

| | | | |
|---|---|---|---|
| Abbr. | Amino Acid | Abbr. | Amino Acid |
| Aad | 2-Aminoadipic acid | EtAsn | N-Ethylasparagine |
| Baad | 3- Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4- Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In certain embodiments, the proteinaceous composition comprises at least one protein, polypeptide or peptide. In further embodiments the proteinaceous composition comprises a biocompatible protein, polypeptide or peptide. As used herein, the term “biocompatible” refers to a substance that produces no significant untoward effects when applied to, or administered to, a given organism (including a mammal, such as a human, dog, cat, horse, and so forth) according to the methods and amounts described herein. Such untoward or undesirable effects are those such as significant toxicity or adverse immunological reactions. In some embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information’s GenBank® and GenPept® databases (ncbi.nlm.nih.gov/). The coding regions for these known genes may be amplified and/or expressed using the techniques disclosed herein or as would be known to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may be fused to another protein, such as fused to at least one antibody. It is contemplated that antibodies to specific tissues may bind the tissue(s) and foster tighter adhesion of the glue to the tissues after welding. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is encompassed herein that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this disclosure may be autologous proteins or peptides, although the disclosure is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being encompassed. The “autologous protein, polypeptide or peptide” may then be used as a component of a composition intended for application to the selected animal or human subject.

In some embodiments of the disclosure a variant of any one of SEQ ID NOs:1-10 has an N-terminal truncation and/or a C-terminal truncation. Any N-terminal truncation and/or C-terminal truncation may be a truncation of exactly or at least or no more than 1, 2, 3, 4, 5, 6, 7, 8, 9, 10, 12, 15, 20, 25, 30, 35, 40, 45, 50, 55, 60, 65, 70, 75, 80, 85, 90, 95, or 100 or more amino acids.

The term “nucleic acid molecule’ or “polynucleotide’ refers to a deoxyribonucleotide or ribonucleotide polymer in either single-stranded or double-stranded form, and, unless specifically indicated otherwise, encompasses polynucleotides containing known analogs of naturally occurring nucleotides that can function in a similar manner as naturally occurring nucleotides. It will be understood that when a nucleic acid molecule is represented by a DNA sequence, this also includes RNA molecules having the corresponding RNA sequence in which “U” (uridine) replaces “T” (thymidine).

Reference to a nucleic acid sequence “encoding” a polypeptide or protein means that the sequence, upon transcription and translation of mRNA, produces the polypeptide. This includes both the coding Strand, whose nucleotide sequence is identical to mRNA and whose sequence is usually provided in the sequence listing, as well as its complementary Strand, which is used as the template for transcription. As any person skilled in the art recognizes, this also includes all degenerate nucleotide sequences encoding the same amino acid sequence. Nucleotide sequences encoding a polypeptide include 25 sequences containing introns.

A biological functional equivalent of any of the proteins may be produced from a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide may be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein’s ability to carry out its designed function. It is thus contemplated by the inventors that various changes may be made in the sequence of genes and proteins disclosed herein, while still fulfilling the goals of the present disclosure.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted.

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

“Conservatively modified variants’ applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant’ where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following six groups each contain amino acids that are conservative substitutions for one another: 1) Alanine (Ala., A), Serine (Ser, S), Threonine (Thr, T); 2) Aspartic acid (Asp, D), Glutamic acid (Glu, E); 3) Asparagine (Asn, N), Glutamine (Gln, Q); 4) Arginine (Arg, R), Lysine (Lys, K); 5) Isoleucine (Ile, I), Leucine (Leu, L), Methionine (Met, M), Valine (Val, V); and 6) Phenylalanine (Phe, F), Tyrosine (Tyr, Y). Tryptophan (Trp, W). In some cases for proteins encompassed in the disclosure, an amino acid within a group has a conservative substitution for another amino acid in the same group.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine ( 0.4); threonine (0.7); serine (0.8); tryptophan (0.9); tyrosine (1.3); proline (1.6); histidine ( 3.2); glutamate (3.5); glutamine (3.5); aspartate (3.5); asparagine (3.5); lysine (3.9); and/or arginine (4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present disclosure. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0 ± 1); glutamate (+3.0 ± 1); serine (+0.3); asparagine (+0.2); glutamine (+0.2); glycine (0); threonine ( 0.4); proline (-0.5 ± 1); alanine (0.5); histidine (0.5); cysteine (1.0); methionine (1.3); valine ( 1.5); leucine (1.8); isoleucine (1.8); tyrosine (2.3); phenylalanine (2.5); tryptophan (3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

The terms “identical” or percent “identity,” in the context of two or more nucleic acids or polypeptide sequences, refer to two or more sequences or subsequences that are the same or have a specified percentage of amino acid residues or nucleotides that are the same as measured using a BLAST or BLAST 2.0 sequence comparison algorithms with default parameters, or by manual alignment and visual inspection (see, e.g., NCBI web site http://www.ncbi.nlm.nih.gov/ BLAST/). Such sequences are then said to be “substantially identical’ or “substantially similar.” The definition also includes sequences that have deletions and/or additions, as well as those that have substitutions. Sequence comparison algorithms can account for gaps and the like.

The term “operatively linked’ or “operably linked” or “operatively joined” or the like, when used to describe “chimeric” or “fusion proteins,” refers to polypeptide sequences that are placed in a physical and functional relationship to each other. In an embodiment, the functions of the polypeptide components of the chimeric molecule are unchanged compared to the functional activities of the parts in isolation. For example, a fluorescent protein of the present disclosure can be fused to a polypeptide of interest. In this case, it is preferable that the fusion molecule retains its fluorescence, and the polypeptide of interest retains its original biological activity. In some embodiments, the activities of either the fluorescent protein or the protein of interest can be reduced relative to their activities in isolation.

As used herein, the term “photostability” refers to a measure of a fluorescent protein’s resistance to the loss of fluorescence upon extended or repeated excitation. Typically, the photostability of a fluorescent protein will be expressed in terms of the photobleaching half-life of the protein, e.g., the time it takes to achieve 50% photobleaching in a homogenous sample of a fluorescent protein. As such, a fluorescent protein variant is considered to have increased or more photostability if the variant has a longer photobleaching half-life as compared to a reference or wild-type fluorescent protein. Fluorescent protein variants having increased photostability may also be referred to herein as “photostable fluorescent protein variants”, “photostable fluorescent proteins,” and the like. Typically, with respect to a variant fluorescent protein, a reference or wild-type fluorescent protein is a fluorescent protein from which said variant is derived, mutated, or evolved from.

The present disclosure provides novel yellow fluorescent protein variants with increased photostability, such as compared to a standard or known or commonly used yellow fluorescent protein. These variants arise from a known yellow fluorescent protein, mVenus (SEQ ID NO:2), which has been mutated or evolved in order to achieve a greater photostability. A yellow fluorescent protein variant having increased photostability may otherwise have identical or highly similar spectral properties to the mVenus reference protein from which it was derived. Alternatively, a photostable fluorescent protein may be a spectral variant or have altered spectral properties with respect to the mVenus reference protein from which it was derived.

In particular embodiments, a composition of any kind comprises, consists of, or consists essentially of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or functional derivatives or functional fragments thereof. The term “functional” as used herein refers to the ability to fluoresce. The fluorescence of the protein of SEQ ID NO:1, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or the functional derivatives or functional fragments thereof, may or may not be measured against another fluorescent protein, such as the fluorescence of the protein of SEQ ID NO:2.

In some embodiments, the present disclosure provides a photostable fluorescent protein of SEQ ID NO:1 comprising amino acid substitutions at residues corresponding to residues 46 and 63, respectively, in wild-type mVenus, In specific embodiments, the amino acid substitutions are L46F and T63S substitutions. In other embodiments, a photostable fluorescent protein comprising the residue 46 and residue 63 substitutions has an amino acid sequence that is at least about 90%, 95%, 98%, or 99% identical to SEQ ID NO:1.

In specific embodiments, a functional derivative of SEQ ID NO:1 has 1, 2, 3, 4, 5, 6, 7, 8, 9, 10, or more amino acid differences compared to SEQ ID NO:1. The amino acid differences may be located anywhere in the polypeptide sequence in SEQ ID NO:1. In specific cases, the difference(s) are in the chromophore region. In specific cases, the difference(s) are in the N-terminal region or the C-terminal region. In specific embodiments, one or more alanines in SEQ ID NO:1 is changed to one of the other amino acids listed above. In specific embodiments, one or more arginines in SEQ ID NO:1 is changed to one of the other amino acids listed above. In specific embodiments, one or more asparagines in SEQ ID NO:1 is changed to one of the other amino acids listed above. In specific embodiments, one or more aspartic acids in SEQ ID NO:1 is changed to one of the other amino acids listed above. In specific embodiments, one or more cysteines in SEQ ID NO:1 is changed to one of the other amino acids listed above. In specific embodiments, one or more glutamines in SEQ ID NO:1 is changed to one of the other amino acids listed above. In specific embodiments, one or more glutamic acids in SEQ ID NO:1 is changed to one of the other amino acids listed above. In specific embodiments, one or more glycines in SEQ ID NO:1 is changed to one of the other amino acids listed above. In specific embodiments, one or more histidines in SEQ ID NO:1 is changed to one of the other amino acids listed above. In specific embodiments, one or more isoleucines in SEQ ID NO:1 is changed to one of the other amino acids listed above. In specific embodiments, one or more lysines in SEQ ID NO:1 is changed to one of the other amino acids listed above. In specific embodiments, one or more methionines in SEQ ID NO:1 is changed to one of the other amino acids listed above. In specific embodiments, one or more phenylalanines in SEQ ID NO:1 is changed to one of the other amino acids listed above. In specific embodiments, one or more phenylalanines in SEQ ID NO:1 is changed to one of the other amino acids listed above. In specific embodiments, one or more prolines in SEQ ID NO:1 is changed to one of the other amino acids listed above. In specific embodiments, one or more serines in SEQ ID NO:1 is changed to one of the other amino acids listed above. In specific embodiments, one or more threonines in SEQ ID NO:1 is changed to one of the other amino acids listed above. In specific embodiments, one or more tryptophans in SEQ ID NO:1 is changed to one of the other amino acids listed above. In specific embodiments, one or more tyrosines in SEQ ID NO:1 is changed to one of the other amino acids listed above. In specific embodiments, one or more valines in SEQ ID NO:1 is changed to one of the other amino acids listed above.

In particular embodiments, functional fragments of SEQ ID NO:1 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

In specific embodiments, a functional derivative of SEQ ID NO:2 has 1, 2, 3, 4, 5, 6, 7, 8, 9, 10, or more amino acid differences compared to SEQ ID NO:2. The amino acid differences may be located anywhere in the polypeptide sequence in SEQ ID NO:2. In specific cases, the difference(s) are in the chromophore region. In specific cases, the difference(s) are in the N-terminal region or the C-terminal region. In specific embodiments, one or more alanines in SEQ ID NO:2 is changed to one of the other amino acids listed above. In specific embodiments, one or more arginines in SEQ ID NO:2 is changed to one of the other amino acids listed above. In specific embodiments, one or more asparagines in SEQ ID NO:2 is changed to one of the other amino acids listed above. In specific embodiments, one or more aspartic acids in SEQ ID NO:2 is changed to one of the other amino acids listed above. In specific embodiments, one or more cysteines in SEQ ID NO:2 is changed to one of the other amino acids listed above. In specific embodiments, one or more glutamines in SEQ ID NO:2 is changed to one of the other amino acids listed above. In specific embodiments, one or more glutamic acids in SEQ ID NO:2 is changed to one of the other amino acids listed above. In specific embodiments, one or more glycines in SEQ ID NO:2 is changed to one of the other amino acids listed above. In specific embodiments, one or more histidines in SEQ ID NO:2 is changed to one of the other amino acids listed above. In specific embodiments, one or more isoleucines in SEQ ID NO:2 is changed to one of the other amino acids listed above. In specific embodiments, one or more lysines in SEQ ID NO:2 is changed to one of the other amino acids listed above. In specific embodiments, one or more methionines in SEQ ID NO:2 is changed to one of the other amino acids listed above. In specific embodiments, one or more phenylalanines in SEQ ID NO:2 is changed to one of the other amino acids listed above. In specific embodiments, one or more phenylalanines in SEQ ID NO:2 is changed to one of the other amino acids listed above. In specific embodiments, one or more prolines in SEQ ID NO:2 is changed to one of the other amino acids listed above. In specific embodiments, one or more serines in SEQ ID NO:2 is changed to one of the other amino acids listed above. In specific embodiments, one or more threonines in SEQ ID NO:2 is changed to one of the other amino acids listed above. In specific embodiments, one or more tryptophans in SEQ ID NO:2 is changed to one of the other amino acids listed above. In specific embodiments, one or more tyrosines in SEQ ID NO:2 is changed to one of the other amino acids listed above. In specific embodiments, one or more valines in SEQ ID NO:2 is changed to one of the other amino acids listed above.

In particular embodiments, functional fragments of SEQ ID NO:2 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:3 comprising an amino acid substitution at a residue corresponding to residue 46 in wild-type mVenus. In one embodiment, the amino acid substitution is an L46F substitution. In other embodiments, a photostable fluorescent protein comprising the residue 46 substitutions has an amino acid sequence that is at least about 90%, 95%, 98%, or 99% identical to SEQ ID NO:3.

In specific embodiments, a functional derivative of SEQ ID NO:3 has 1, 2, 3, 4, 5, 6, 7, 8, 9, 10, or more amino acid differences compared to SEQ ID NO:3. The amino acid differences may be located anywhere in the polypeptide sequence in SEQ ID NO:3. In specific cases, the difference(s) are in the chromophore region. In specific cases, the difference(s) are in the N-terminal region or the C-terminal region. In specific embodiments, one or more alanines in SEQ ID NO:3 is changed to one of the other amino acids listed above. In specific embodiments, one or more arginines in SEQ ID NO:3 is changed to one of the other amino acids listed above. In specific embodiments, one or more asparagines in SEQ ID NO:3 is changed to one of the other amino acids listed above. In specific embodiments, one or more aspartic acids in SEQ ID NO:3 is changed to one of the other amino acids listed above. In specific embodiments, one or more cysteines in SEQ ID NO:3 is changed to one of the other amino acids listed above. In specific embodiments, one or more glutamines in SEQ ID NO:3 is changed to one of the other amino acids listed above. In specific embodiments, one or more glutamic acids in SEQ ID NO:3 is changed to one of the other amino acids listed above. In specific embodiments, one or more glycines in SEQ ID NO:3 is changed to one of the other amino acids listed above. In specific embodiments, one or more histidines in SEQ ID NO:3 is changed to one of the other amino acids listed above. In specific embodiments, one or more isoleucines in SEQ ID NO:3 is changed to one of the other amino acids listed above. In specific embodiments, one or more lysines in SEQ ID NO:3 is changed to one of the other amino acids listed above. In specific embodiments, one or more methionines in SEQ ID NO:3 is changed to one of the other amino acids listed above. In specific embodiments, one or more phenylalanines in SEQ ID NO:3 is changed to one of the other amino acids listed above. In specific embodiments, one or more phenylalanines in SEQ ID NO:3 is changed to one of the other amino acids listed above. In specific embodiments, one or more prolines in SEQ ID NO:3 is changed to one of the other amino acids listed above. In specific embodiments, one or more serines in SEQ ID NO:3 is changed to one of the other amino acids listed above. In specific embodiments, one or more threonines in SEQ ID NO:3 is changed to one of the other amino acids listed above. In specific embodiments, one or more tryptophans in SEQ ID NO:3 is changed to one of the other amino acids listed above. In specific embodiments, one or more tyrosines in SEQ ID NO:3 is changed to one of the other amino acids listed above. In specific embodiments, one or more valines in SEQ ID NO:3 is changed to one of the other amino acids listed above.

In particular embodiments, functional fragments of SEQ ID NO:3 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:4 comprising amino acid substitutions at residues corresponding to residues 46, 47, and 48, respectively, in wild-type mVenus, In specific embodiments, the amino acid substitutions are L46F, 147V and C48L substitutions. In other embodiments, a photostable fluorescent protein comprising the residue 46, residue 47, and residue 48 substitutions has an amino acid sequence that is at least about 90%, 95%, 98%, or 99% identical to SEQ ID NO:4.

In specific embodiments, a functional derivative of SEQ ID NO:4 has 1, 2, 3, 4, 5, 6, 7, 8, 9, 10, or more amino acid differences compared to SEQ ID NO:4. The amino acid differences may be located anywhere in the polypeptide sequence in SEQ ID NO:4. In specific cases, the difference(s) are in the chromophore region. In specific cases, the difference(s) are in the N-terminal region or the C-terminal region. In specific embodiments, one or more alanines in SEQ ID NO:4 is changed to one of the other amino acids listed above. In specific embodiments, one or more arginines in SEQ ID NO:4 is changed to one of the other amino acids listed above. In specific embodiments, one or more asparagines in SEQ ID NO:4 is changed to one of the other amino acids listed above. In specific embodiments, one or more aspartic acids in SEQ ID NO:4 is changed to one of the other amino acids listed above. In specific embodiments, one or more cysteines in SEQ ID NO:4 is changed to one of the other amino acids listed above. In specific embodiments, one or more glutamines in SEQ ID NO:4 is changed to one of the other amino acids listed above. In specific embodiments, one or more glutamic acids in SEQ ID NO:4 is changed to one of the other amino acids listed above. In specific embodiments, one or more glycines in SEQ ID NO:4 is changed to one of the other amino acids listed above. In specific embodiments, one or more histidines in SEQ ID NO:4 is changed to one of the other amino acids listed above. In specific embodiments, one or more isoleucines in SEQ ID NO:4 is changed to one of the other amino acids listed above. In specific embodiments, one or more lysines in SEQ ID NO:4 is changed to one of the other amino acids listed above. In specific embodiments, one or more methionines in SEQ ID NO:1 is changed to one of the other amino acids listed above. In specific embodiments, one or more phenylalanines in SEQ ID NO:4 is changed to one of the other amino acids listed above. In specific embodiments, one or more phenylalanines in SEQ ID NO:4 is changed to one of the other amino acids listed above. In specific embodiments, one or more prolines in SEQ ID NO:4 is changed to one of the other amino acids listed above. In specific embodiments, one or more serines in SEQ ID NO:4 is changed to one of the other amino acids listed above. In specific embodiments, one or more threonines in SEQ ID NO:4 is changed to one of the other amino acids listed above. In specific embodiments, one or more tryptophans in SEQ ID NO:4 is changed to one of the other amino acids listed above. In specific embodiments, one or more tyrosines in SEQ ID NO:4 is changed to one of the other amino acids listed above. In specific embodiments, one or more valines in SEQ ID NO:4 is changed to one of the other amino acids listed above.

In particular embodiments, functional fragments of SEQ ID NO:4 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:5 comprising amino acid substitutions at residues corresponding to residues 204, 205, and 206, respectively, in wild-type mVenus, In specific embodiments, the amino acid substitutions are Q204N, S205A, and K206S substitutions. In other embodiments, a photostable fluorescent protein comprising the residue 204, residue 205, and residue 206 substitutions has an amino acid sequence that is at least about 90%, 95%, 98%, or 99% identical to SEQ ID NO:5.

In specific embodiments, a functional derivative of SEQ ID NO:5 has 1, 2, 3, 4, 5, 6, 7, 8, 9, 10, or more amino acid differences compared to SEQ ID NO:5. The amino acid differences may be located anywhere in the polypeptide sequence in SEQ ID NO:5. In specific cases, the difference(s) are in the chromophore region. In specific cases, the difference(s) are in the N-terminal region or the C-terminal region. In specific embodiments, one or more alanines in SEQ ID NO:5 is changed to one of the other amino acids listed above. In specific embodiments, one or more arginines in SEQ ID NO:5 is changed to one of the other amino acids listed above. In specific embodiments, one or more asparagines in SEQ ID NO:5 is changed to one of the other amino acids listed above. In specific embodiments, one or more aspartic acids in SEQ ID NO:5 is changed to one of the other amino acids listed above. In specific embodiments, one or more cysteines in SEQ ID NO:5 is changed to one of the other amino acids listed above. In specific embodiments, one or more glutamines in SEQ ID NO:5 is changed to one of the other amino acids listed above. In specific embodiments, one or more glutamic acids in SEQ ID NO:5 is changed to one of the other amino acids listed above. In specific embodiments, one or more glycines in SEQ ID NO:5 is changed to one of the other amino acids listed above. In specific embodiments, one or more histidines in SEQ ID NO:5 is changed to one of the other amino acids listed above. In specific embodiments, one or more isoleucines in SEQ ID NO:5 is changed to one of the other amino acids listed above. In specific embodiments, one or more lysines in SEQ ID NO:5 is changed to one of the other amino acids listed above. In specific embodiments, one or more methionines in SEQ ID NO:5 is changed to one of the other amino acids listed above. In specific embodiments, one or more phenylalanines in SEQ ID NO:5 is changed to one of the other amino acids listed above. In specific embodiments, one or more phenylalanines in SEQ ID NO:5 is changed to one of the other amino acids listed above. In specific embodiments, one or more prolines in SEQ ID NO:5 is changed to one of the other amino acids listed above. In specific embodiments, one or more serines in SEQ ID NO:5 is changed to one of the other amino acids listed above. In specific embodiments, one or more threonines in SEQ ID NO:5 is changed to one of the other amino acids listed above. In specific embodiments, one or more tryptophans in SEQ ID NO:5is changed to one of the other amino acids listed above. In specific embodiments, one or more tyrosines in SEQ ID NO:5 is changed to one of the other amino acids listed above. In specific embodiments, one or more valines in SEQ ID NO:5 is changed to one of the other amino acids listed above.

In particular embodiments, functional fragments of SEQ ID NO:5 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:6 comprising an amino acid substitution at a residue corresponding to residues 46, 63, and 163, respectively, in wild-type mVenus. In one embodiment, the amino acid substitutions are an L46F, T63S, and A163V substitutions. In other embodiments, a photostable fluorescent protein comprising the residue 46, residue 63, and residue 163 substitutions has an amino acid sequence that is at least about 90%, 95%, 98%, or 99% identical to SEQ ID NO:6.

In specific embodiments, a functional derivative of SEQ ID NO:6 has 1, 2, 3, 4, 5, 6, 7, 8, 9, 10, or more amino acid differences compared to SEQ ID NO:6. The amino acid differences may be located anywhere in the polypeptide sequence in SEQ ID NO:6. In specific cases, the difference(s) are in the chromophore region. In specific cases, the difference(s) are in the N-terminal region or the C-terminal region. In specific embodiments, one or more alanines in SEQ ID NO:6 is changed to one of the other amino acids listed above. In specific embodiments, one or more arginines in SEQ ID NO:6 is changed to one of the other amino acids listed above. In specific embodiments, one or more asparagines in SEQ ID NO:6 is changed to one of the other amino acids listed above. In specific embodiments, one or more aspartic acids in SEQ ID NO:6 is changed to one of the other amino acids listed above. In specific embodiments, one or more cysteines in SEQ ID NO:6 is changed to one of the other amino acids listed above. In specific embodiments, one or more glutamines in SEQ ID NO:6 is changed to one of the other amino acids listed above. In specific embodiments, one or more glutamic acids in SEQ ID NO:6 is changed to one of the other amino acids listed above. In specific embodiments, one or more glycines in SEQ ID NO:6 is changed to one of the other amino acids listed above. In specific embodiments, one or more histidines in SEQ ID NO:6 is changed to one of the other amino acids listed above. In specific embodiments, one or more isoleucines in SEQ ID NO:6 is changed to one of the other amino acids listed above. In specific embodiments, one or more lysines in SEQ ID NO:6 is changed to one of the other amino acids listed above. In specific embodiments, one or more methionines in SEQ ID NO:6 is changed to one of the other amino acids listed above. In specific embodiments, one or more phenylalanines in SEQ ID NO:6 is changed to one of the other amino acids listed above. In specific embodiments, one or more phenylalanines in SEQ ID NO:6 is changed to one of the other amino acids listed above. In specific embodiments, one or more prolines in SEQ ID NO:6 is changed to one of the other amino acids listed above. In specific embodiments, one or more serines in SEQ ID NO:6 is changed to one of the other amino acids listed above. In specific embodiments, one or more threonines in SEQ ID NO:6 is changed to one of the other amino acids listed above. In specific embodiments, one or more tryptophans in SEQ ID NO:6 is changed to one of the other amino acids listed above. In specific embodiments, one or more tyrosines in SEQ ID NO:6 is changed to one of the other amino acids listed above. In specific embodiments, one or more valines in SEQ ID NO:6 is changed to one of the other amino acids listed above.

In particular embodiments, functional fragments of SEQ ID NO:6 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:7 comprising an amino acid substitution at a residue corresponding to residues 46, 63, 80, 147, and 232, respectively, in wild-type mVenus. In one embodiment, the amino acid substitutions are an L46F, T63S, Q80R, S147C, and G232S substitutions. In other embodiments, a photostable fluorescent protein comprising the residue 46, residue 63, residue 80, residue 147, and 232 substitutions has an amino acid sequence that is at least about 90%, 95%, 98%, or 99% identical to SEQ ID NO:7.

In specific embodiments, a functional derivative of SEQ ID NO:7 has 1, 2, 3, 4, 5, 6, 7, 8, 9, 10, or more amino acid differences compared to SEQ ID NO:7. The amino acid differences may be located anywhere in the polypeptide sequence in SEQ ID NO:7. In specific cases, the difference(s) are in the chromophore region. In specific cases, the difference(s) are in the N-terminal region or the C-terminal region. In specific embodiments, one or more alanines in SEQ ID NO:7 is changed to one of the other amino acids listed above. In specific embodiments, one or more arginines in SEQ ID NO:7 is changed to one of the other amino acids listed above. In specific embodiments, one or more asparagines in SEQ ID NO:7 is changed to one of the other amino acids listed above. In specific embodiments, one or more aspartic acids in SEQ ID NO:7 is changed to one of the other amino acids listed above. In specific embodiments, one or more cysteines in SEQ ID NO:7 is changed to one of the other amino acids listed above. In specific embodiments, one or more glutamines in SEQ ID NO:7 is changed to one of the other amino acids listed above. In specific embodiments, one or more glutamic acids in SEQ ID NO:7 is changed to one of the other amino acids listed above. In specific embodiments, one or more glycines in SEQ ID NO:7 is changed to one of the other amino acids listed above. In specific embodiments, one or more histidines in SEQ ID NO:7 is changed to one of the other amino acids listed above. In specific embodiments, one or more isoleucines in SEQ ID NO:7 is changed to one of the other amino acids listed above. In specific embodiments, one or more lysines in SEQ ID NO:7 is changed to one of the other amino acids listed above. In specific embodiments, one or more methionines in SEQ ID NO:7 is changed to one of the other amino acids listed above. In specific embodiments, one or more phenylalanines in SEQ ID NO:7 is changed to one of the other amino acids listed above. In specific embodiments, one or more phenylalanines in SEQ ID NO:7 is changed to one of the other amino acids listed above. In specific embodiments, one or more prolines in SEQ ID NO:7 is changed to one of the other amino acids listed above. In specific embodiments, one or more serines in SEQ ID NO:7 is changed to one of the other amino acids listed above. In specific embodiments, one or more threonines in SEQ ID NO:7 is changed to one of the other amino acids listed above. In specific embodiments, one or more tryptophans in SEQ ID NO:7 is changed to one of the other amino acids listed above. In specific embodiments, one or more tyrosines in SEQ ID NO:7 is changed to one of the other amino acids listed above. In specific embodiments, one or more valines in SEQ ID NO:7 is changed to one of the other amino acids listed above.

In particular embodiments, functional fragments of SEQ ID NO:7 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:8 comprising an amino acid substitution at a residue corresponding to residues 46, 63, 147, and 156, respectively, in wild-type mVenus. In one embodiment, the amino acid substitutions are an L46F, T63S, S147N, and K156R substitutions. In other embodiments, a photostable fluorescent protein comprising the residue 46, residue 63, residue 147, and residue 156 substitutions has an amino acid sequence that is at least about 90%, 95%, 98%, or 99% identical to SEQ ID NO:8.

In specific embodiments, a functional derivative of SEQ ID NO:8 has 1, 2, 3, 4, 5, 6, 7, 8, 9, 10, or more amino acid differences compared to SEQ ID NO:8. The amino acid differences may be located anywhere in the polypeptide sequence in SEQ ID NO:8. In specific cases, the difference(s) are in the chromophore region. In specific cases, the difference(s) are in the N-terminal region or the C-terminal region. In specific embodiments, one or more alanines in SEQ ID NO:8 is changed to one of the other amino acids listed above. In specific embodiments, one or more arginines in SEQ ID NO:8 is changed to one of the other amino acids listed above. In specific embodiments, one or more asparagines in SEQ ID NO:8 is changed to one of the other amino acids listed above. In specific embodiments, one or more aspartic acids in SEQ ID NO:8 is changed to one of the other amino acids listed above. In specific embodiments, one or more cysteines in SEQ ID NO:8 is changed to one of the other amino acids listed above. In specific embodiments, one or more glutamines in SEQ ID NO:8 is changed to one of the other amino acids listed above. In specific embodiments, one or more glutamic acids in SEQ ID NO:8 is changed to one of the other amino acids listed above. In specific embodiments, one or more glycines in SEQ ID NO:8 is changed to one of the other amino acids listed above. In specific embodiments, one or more histidines in SEQ ID NO:8 is changed to one of the other amino acids listed above. In specific embodiments, one or more isoleucines in SEQ ID NO:8 is changed to one of the other amino acids listed above. In specific embodiments, one or more lysines in SEQ ID NO:8 is changed to one of the other amino acids listed above. In specific embodiments, one or more methionines in SEQ ID NO:8 is changed to one of the other amino acids listed above. In specific embodiments, one or more phenylalanines in SEQ ID NO:8 is changed to one of the other amino acids listed above. In specific embodiments, one or more phenylalanines in SEQ ID NO:8 is changed to one of the other amino acids listed above. In specific embodiments, one or more prolines in SEQ ID NO:8 is changed to one of the other amino acids listed above. In specific embodiments, one or more serines in SEQ ID NO:8 is changed to one of the other amino acids listed above. In specific embodiments, one or more threonines in SEQ ID NO:8 is changed to one of the other amino acids listed above. In specific embodiments, one or more tryptophans in SEQ ID NO:8 is changed to one of the other amino acids listed above. In specific embodiments, one or more tyrosines in SEQ ID NO:8 is changed to one of the other amino acids listed above. In specific embodiments, one or more valines in SEQ ID NO:8 is changed to one of the other amino acids listed above.

In particular embodiments, functional fragments of SEQ ID NO:8 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:9 comprising an amino acid substitution at a residue corresponding to residues 46, 63, and 78, respectively, in wild-type mVenus. In one embodiment, the amino acid substitutions are an L46F, T63S, and M78L substitutions. In other embodiments, a photostable fluorescent protein comprising the residue 46, residue 63, and residue 78 substitutions has an amino acid sequence that is at least about 90%, 95%, 98%, or 99% identical to SEQ ID NO:9.

In specific embodiments, a functional derivative of SEQ ID NO:9 has 1, 2, 3, 4, 5, 6, 7, 8, 9, 10, or more amino acid differences compared to SEQ ID NO:9. The amino acid differences may be located anywhere in the polypeptide sequence in SEQ ID NO:9. In specific cases, the difference(s) are in the chromophore region. In specific cases, the difference(s) are in the N-terminal region or the C-terminal region. In specific embodiments, one or more alanines in SEQ ID NO:9 is changed to one of the other amino acids listed above. In specific embodiments, one or more arginines in SEQ ID NO:9 is changed to one of the other amino acids listed above. In specific embodiments, one or more asparagines in SEQ ID NO:9 is changed to one of the other amino acids listed above. In specific embodiments, one or more aspartic acids in SEQ ID NO:9 is changed to one of the other amino acids listed above. In specific embodiments, one or more cysteines in SEQ ID NO:9 is changed to one of the other amino acids listed above. In specific embodiments, one or more glutamines in SEQ ID NO:9 is changed to one of the other amino acids listed above. In specific embodiments, one or more glutamic acids in SEQ ID NO:9 is changed to one of the other amino acids listed above. In specific embodiments, one or more glycines in SEQ ID NO:9 is changed to one of the other amino acids listed above. In specific embodiments, one or more histidines in SEQ ID NO:9 is changed to one of the other amino acids listed above. In specific embodiments, one or more isoleucines in SEQ ID NO:9 is changed to one of the other amino acids listed above. In specific embodiments, one or more lysines in SEQ ID NO:9 is changed to one of the other amino acids listed above. In specific embodiments, one or more methionines in SEQ ID NO:9 is changed to one of the other amino acids listed above. In specific embodiments, one or more phenylalanines in SEQ ID NO:9 is changed to one of the other amino acids listed above. In specific embodiments, one or more phenylalanines in SEQ ID NO:9 is changed to one of the other amino acids listed above. In specific embodiments, one or more prolines in SEQ ID NO:9 is changed to one of the other amino acids listed above. In specific embodiments, one or more serines in SEQ ID NO:9 is changed to one of the other amino acids listed above. In specific embodiments, one or more threonines in SEQ ID NO:9 is changed to one of the other amino acids listed above. In specific embodiments, one or more tryptophans in SEQ ID NO:9 is changed to one of the other amino acids listed above. In specific embodiments, one or more tyrosines in SEQ ID NO:9 is changed to one of the other amino acids listed above. In specific embodiments, one or more valines in SEQ ID NO:9 is changed to one of the other amino acids listed above.

In particular embodiments, functional fragments of SEQ ID NO:9 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids.

Some embodiments provide a photostable fluorescent protein of SEQ ID NO:10 comprising an amino acid substitution at a residue corresponding to residues 46, 63, and 151, respectively, in wild-type mVenus. In one embodiment, the amino acid substitutions are an L46F, T63S, and Y151C substitutions. In other embodiments, a photostable fluorescent protein comprising the residue 46, residue 63, and residue 151 substitutions has an amino acid sequence that is at least about 90%, 95%, 98%, or 99% identical to SEQ ID NO:6.

In specific embodiments, a functional derivative of SEQ ID NO:10 has 1, 2, 3, 4, 5, 6, 7, 8, 9, 10, or more amino acid differences compared to SEQ ID NO:10. The amino acid differences may be located anywhere in the polypeptide sequence in SEQ ID NO:10. In specific cases, the difference(s) are in the chromophore region. In specific cases, the difference(s) are in the N-terminal region or the C-terminal region. In specific embodiments, one or more alanines in SEQ ID NO:10 is changed to one of the other amino acids listed above. In specific embodiments, one or more arginines in SEQ ID NO:10 is changed to one of the other amino acids listed above. In specific embodiments, one or more asparagines in SEQ ID NO:10 is changed to one of the other amino acids listed above. In specific embodiments, one or more aspartic acids in SEQ ID NO:10 is changed to one of the other amino acids listed above. In specific embodiments, one or more cysteines in SEQ ID NO:10 is changed to one of the other amino acids listed above. In specific embodiments, one or more glutamines in SEQ ID NO:10 is changed to one of the other amino acids listed above. In specific embodiments, one or more glutamic acids in SEQ ID NO:10 is changed to one of the other amino acids listed above. In specific embodiments, one or more glycines in SEQ ID NO:10 is changed to one of the other amino acids listed above. In specific embodiments, one or more histidines in SEQ ID NO:10 is changed to one of the other amino acids listed above. In specific embodiments, one or more isoleucines in SEQ ID NO:10 is changed to one of the other amino acids listed above. In specific embodiments, one or more lysines in SEQ ID NO:10 is changed to one of the other amino acids listed above. In specific embodiments, one or more methionines in SEQ ID NO:10 is changed to one of the other amino acids listed above. In specific embodiments, one or more phenylalanines in SEQ ID NO:10 is changed to one of the other amino acids listed above. In specific embodiments, one or more phenylalanines in SEQ ID NO:10 is changed to one of the other amino acids listed above. In specific embodiments, one or more prolines in SEQ ID NO:10 is changed to one of the other amino acids listed above. In specific embodiments, one or more serines in SEQ ID NO:10 is changed to one of the other amino acids listed above. In specific embodiments, one or more threonines in SEQ ID NO:10 is changed to one of the other amino acids listed above. In specific embodiments, one or more tryptophans in SEQ ID NO:10 is changed to one of the other amino acids listed above. In specific embodiments, one or more tyrosines in SEQ ID NO:10 is changed to one of the other amino acids listed above. In specific embodiments, one or more valines in SEQ ID NO:10 is changed to one of the other amino acids listed above.

In particular embodiments, functional fragments of SEQ ID NO:10 are utilized as compositions and in methods of any kind. The fragment may be of any length, but in specific embodiments the fragment is of the following length of amino acids or is less than the following length of amino acids: 238, 237, 236, 235, 234, 233, 232, 231, 230, 229, 228, 227, 226, 225, 224, 223, 222, 221, 220, 219, 218, 217, 216, 215, 214, 213, 212, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 145, 140, 135, 130, 125, 120, 115, 110, 105, 100, 95, 90, 85, 80, 75, 70, 65, 60, 55, 50, 45, 40, 35, 30, 25, 20, 15, of 10 amino acids

Some embodiments of the disclosure are fusion proteins comprising any protein of interest operatively joined to at least one photostable fluorescent protein variant. This fusion protein can optionally contain a peptide tag. For example, a polyhistidine tag containing, for example, six histidine residues, can be incorporated at the N-Terminus or C-Terminus of the fluorescent protein variant, which then can be isolated in a single step using nickel-chelate chromatography. Additional non-limiting examples of a peptide tag includes a GST tag, a c-myc peptide, a FLAG epitope, or any ligand (or cognate receptor), and a peptide epitope.

Some embodiments are tandem fluorescent proteins that are competent for fluorescence resonance energy transfer (FRET). These proteins comprise two fluorescent proteins operatively linked by a peptide linker, wherein at least one of the fluorescent proteins is a photostable fluorescent protein variant.

In some embodiments, nucleic acid molecules are provided that encode for photostable fluorescent protein variants. In specific embodiments, the nucleic acid molecules are found in vectors. In certain embodiments, the nucleic acid molecules are functionally linked to a regulatory control element, such as a promoter or enhancer sequence. In specific embodiments, nucleic acid molecules include at least one polynucleotide encoding a photostable fluorescent protein variant operatively linked to one or more other polynucleotides. The one or more other polynucleotides can be, for example, a transcription regulatory element such as a promoter or polyadenylation signal sequence, or a translation regulatory element such as a ribosome binding site. Such a recombinant nucleic acid molecule can be contained in a vector, which can be an expression vector, and the nucleic acid molecule or the vector can be contained in a host cell. The vector generally contains elements required for replication in a prokaryotic or eukaryotic host system or both, as desired. In some embodiments, the vectors include plasmid vectors and viral vectors such as bacteriophage, baculovirus, retrovirus, lentivirus, adenovirus, vaccinia virus, semliki forest virus and adeno-associated virus vectors. In some embodiments, the vector is an expression vector that contains an expression control sequence operatively linked to a photostable fluorescent protein variant, as indicated above. The expression vector can be adapted for function in prokaryotes or eukaryotes by inclusion of appropriate promoters, replication sequences, markers, and the like. An expression vector can be transfected into a recombinant host cell for expression of photostable fluorescent protein variant, and host cells can be selected, for example, for high levels of expression in order to obtain a large amount of isolated protein. A host cell can be maintained in cell culture, or can be a cell in vivo in an organism.

In some embodiments, the present disclosure provides host cells comprising a photostable fluorescent protein variant or polynucleotide encoding a photostable fluorescent protein variant. Suitable host cells include, without limitation, bacteria, yeasts, fungi, and animal and plant cells. Non-limiting examples of suitable prokaryotic host cells include a strain of E. coli, a strain of Enterobacter, a strain of Salmonella, a strain of Bacilli, such as B. subtilis or B. licheniformis, a strain of Pseudomonas, a strain of Streptomyces, and the like. Non-limiting examples of eukaryotic host cells include without limitation, a yeast, such as Saccharomyces cerevisiae, Schizosaccharomyces pombe, or a Kluyveromyces yeast, Neurospora crassa, a fungus or mold, such as Neurospora, Penicillium, Tolypocladium, Aspergillus, an insect cell, such as a Drosophilai cell or an Anopheles cell, a mammalian cell, such as a CHO cell, a COS cell, a human cell, a 293 cell, a HeLa cell, a Hep G2 cell, a mouse cell, and the like.

Some embodiments provide methods of detecting the expression of a protein using a photostable fluorescent protein variant or a nucleic acid encoding a photostable fluorescent protein variant of the disclosure. In one embodiment, the method comprises the steps of expressing a fusion protein comprising a photostable fluorescent protein variant and a target protein of interest in a cell or cellular extract, and detecting the fluorescence of the fluorescent protein variant, thereby detecting the expression of a target protein of interest. In other embodiments, the method comprises the steps of expressing a fusion protein comprising a photostable fluorescent protein variant of the disclosure and a peptide or protein that binds to a target protein of interest in a cell or a cellular extract, and detecting a difference in fluorescence or a property related to fluorescence, such as relative or total fluorescence, fluorescence anisotropy or fluorescence polarization, thereby detecting expression of a target protein of interest.

Some embodiments provide a method of detecting the localization of a protein using a photostable fluorescent protein variant or a nucleic acid encoding a photostable fluorescent protein variant. In one embodiment, the method comprises the steps of expressing a fusion protein comprising a photostable fluorescent protein variant and a target protein of interest in a cell, detecting the fluorescence of the photostable fluorescent protein variant, and determining the cellular location of the fluorescence, thereby determining the localization of the target protein of interest.

Other embodiments provide a method of detecting protein motility using a photostable fluorescent protein variant or a nucleic acid encoding a photostable fluorescent protein variant of the disclosure is provided. In one embodiment, the method comprises the steps of expressing a fusion protein comprising a photostable fluorescent protein variant of the disclosure and a target protein of interest in a cell, performing time-sequential observations of fluorescence in said cell, and detecting differences in fluorescence between said time-sequential observations, thereby detecting protein motility of a target protein of interest.

Some embodiments provide a method of detecting a protein-protein interaction using a photostable fluorescent protein variant or a nucleic acid encoding a photostable fluorescent protein variant of the disclosure. In particular embodiments, the method comprises the steps of contacting a fusion protein comprising a photostable fluorescent protein variant of the disclosure and a first protein of interest with a second protein of interest, and detecting a change in fluorescence or a change in a property related to fluorescence, thereby detecting an interaction between a first protein of interest and a second protein of interest. In other embodiments, the second protein of interest comprises a fusion protein of the second protein of interest and a fluorescent protein. In certain embodiments, said second fluorescent protein may be a photostable fluorescent protein variant of the disclosure. Protein-protein interactions may be detected by measuring FRET between two suitable fluorescent proteins, such as measuring relative fluorescence, fluorescence anisotropy, or fluorescence polarization. Protein-protein interactions may be measured in vivo, in vitro, ex vivo, in a cell, in a cellular extract, and the like.

A photostable fluorescent protein variant of the disclosure is useful in any method that employs a fluorescent protein. Thus, the photostable fluorescent protein variants are useful as fluorescent markers in the many ways fluorescent markers already are used, including, for example, coupling photostable fluorescent protein variants to antibodies, polynucleotides, or other receptors for use in detection assays such as immunoassays or hybridization assays, or to track the movement of proteins in cells. For intracellular tracking studies, a first (or other) polynucleotide encoding the fluorescent protein variant is fused to a second (or other) polynucleotide encoding a protein of interest and the construct, if desired, can be inserted into an expression vector. Upon expression inside the cell, the protein of interest can be localized based on fluorescence, without concern that localization of the protein is an artifact caused by oligomerization of the fluorescent protein component of the fusion protein. In one embodiment of this method, two proteins of interest independently are fused with two fluorescent protein variants that have different fluorescent characteristics.

The photostable fluorescent protein variants of this disclosure are useful in systems to detect induction of transcription. For example, a nucleotide sequence encoding a photostable fluorescent proteins variant can be fused to a promoter or other expression control sequence of interest, which can be contained in an expression vector, the construct can be transfected into a cell, and induction of the promoter (or other regulatory element) can be measured by detecting the presence or amount of fluorescence, thereby allowing a means to observe the responsiveness of a signaling pathway from receptor to promoter.

A photostable fluorescent protein variant of the disclosure also is useful in applications involving FRET, which can detect events as a function of the movement of fluorescent donors and acceptors towards or away from each other. One or both of the donor/acceptor pair can be a photostable fluorescent protein variant. Such a donor/acceptor pair provides a wide separation between the excitation and emission peaks of the donor, and provides good overlap between the donor emission spectrum and the acceptor excitation spectrum.

Fluorescence in a sample can be measured using a fluorimeter or a microscope, wherein excitation radiation from an excitation source having a first wavelength, passes through excitation optics, which cause the excitation radiation to excite the sample. In response, a photostable fluorescent protein variant in the sample emits radiation having a wavelength that is different from the excitation wavelength. Collection optics then collect the emission from the sample. The device can include a temperature controller to maintain the sample at a specific temperature while it is being scanned, and can have a multi-axis translation stage, which moves a microtiter plate holding a plurality of samples in order to position different wells to be exposed. The multi-axis translation stage, temperature controller, autofocusing feature, and electronics associated with imaging and data collection can be managed by an appropriately programmed digital computer, which also can transform the data collected during the assay into another format for presentation. This process can be miniaturized and automated to enable screening many thousands of compounds in a high throughput format. These and other methods of performing assays on fluorescent materials are well known in the art (see, e.g., Lakowicz, “Principles of Fluorescence Spectroscopy” (Plenum Press 1983); Herman, Meth. Cell Biol., 30:219-243 (1989); Turro, “Modern Molecular Photochemistry” (Benjamin/Cummings Publ. Co., Inc., 1978), pp. 296-361, each of which is incorporated herein by reference).

The sample to be examined can be any sample, including a biological sample, an environmental sample, or any other sample for which it is desired to determine whether a particular molecule is present therein. Preferably, the sample includes a cell or an extract thereof. The cell can be obtained from a vertebrate, including a mammal such as a human, or from an invertebrate, and can be a cell from a plant or an animal. The cell can be obtained from a culture of such cells, for example, a cell line, or can be isolated from an organism. As such, the cell can be contained in a tissue sample, which can be obtained from an organism by any means commonly used to obtain a tissue sample, for example, by biopsy of a human. Where the method is performed using an intact living cell or a freshly isolated tissue or organ sample, the presence of a molecule of interest in living cells can be identified, thus providing a means to determine, for example, the intracellular compartmentalization of the molecule.

A photostable fluorescent protein variant can be linked to the molecule directly or indirectly, using any linkage that is stable under the conditions to which the protein-molecule complex is to be exposed. Thus, the fluorescent protein and molecule can be linked via a chemical reaction between reactive groups present on the protein and molecule, or the linkage can be mediated by linker moiety, which contains reactive groups specific for the fluorescent protein and the molecule. It will be recognized that the appropriate conditions for linking the fluorescent protein variant and the molecule are selected depending, for example, on the chemical nature of the molecule and the type of linkage desired. Where the molecule of interest is a polypeptide, a convenient means for linking a photostable fluorescent protein variant and the molecule is by expressing them as a fusion protein from a recombinant nucleic acid molecule, which comprises a polynucleotide encoding, for example, a tandem fluorescent protein operatively linked to a polynucleotide encoding the polypeptide molecule.

### EXAMPLES

### Example 1. Identifying Photostable mVenus Variants

### Selecting Residues for Mutagenesis

The residues of mVenus selected for modification were primarily chosen based on the likely effect of the residue on photostability or conformational flexibility of the chromophore.

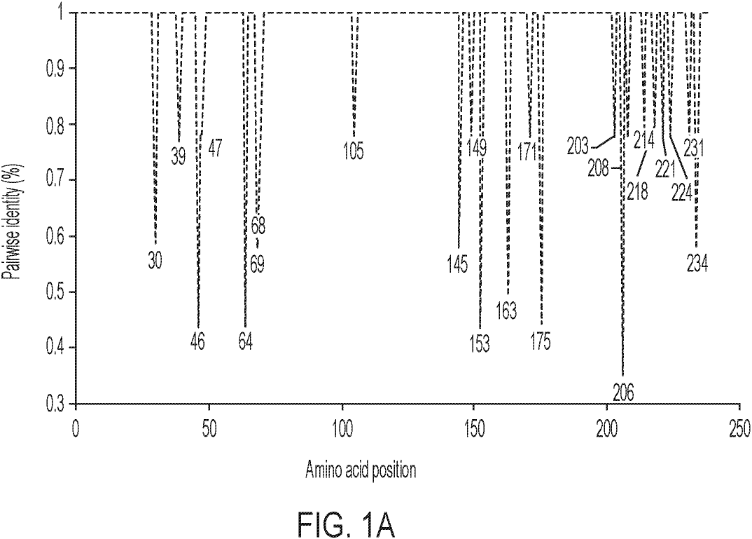

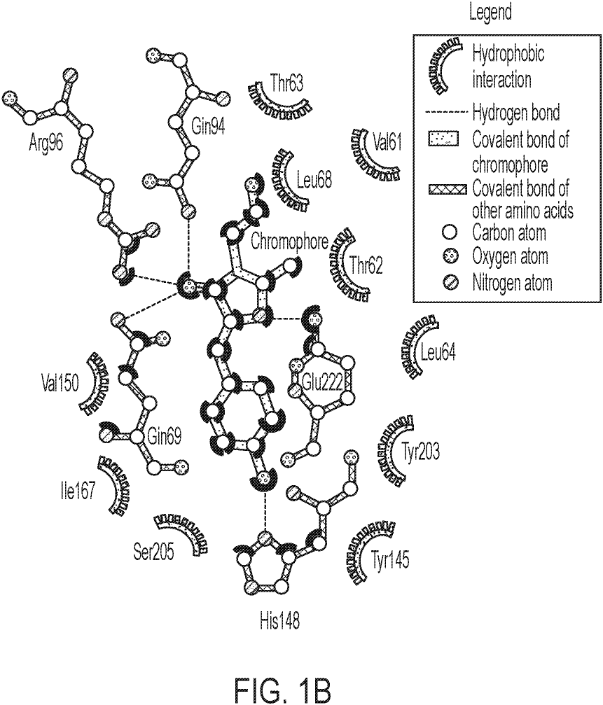

FIG. 1A is a chart showing residues with large variations between engineered YFPs. First, protein sequences of 9 YFPs (mVenus, Venus, SYFP2, moxVenus, SHardonnay, EYFP, mCitrine, and Citrine2) were aligned using global alignment with free end gaps and the Blosum62 scoring matrix. The pairwise identity percentage was then computed by dividing the number of identical pairs by the total number of pairs. The numbers below the peaks correspond to the position of residues with low pairwise identity and red font was used to denote the residues mutated in this study. Several chosen residues were prioritized due to ease of cloning (e.g. mutations located nearby can be easily combined on the same primer).

FIG. 1B is a graphic showing residues selected for mutagenesis that interact with the chromophore of mVenus. LigPlot+ was used to identify residues making hydrophobic or hydrogen bond interactions with the chromophore using the crystal structure of Venus (PDB: 1MYW). The amino acids labeled in red were mutagenized in this study. Several chosen residues were prioritized due to ease of cloning (e.g. mutations located close by can be easily combined on the same primer).

Overall, 21 different residues of mVenus were targeted.

### Screening Mvenus Variants by Monitoring Photostability and Brightness

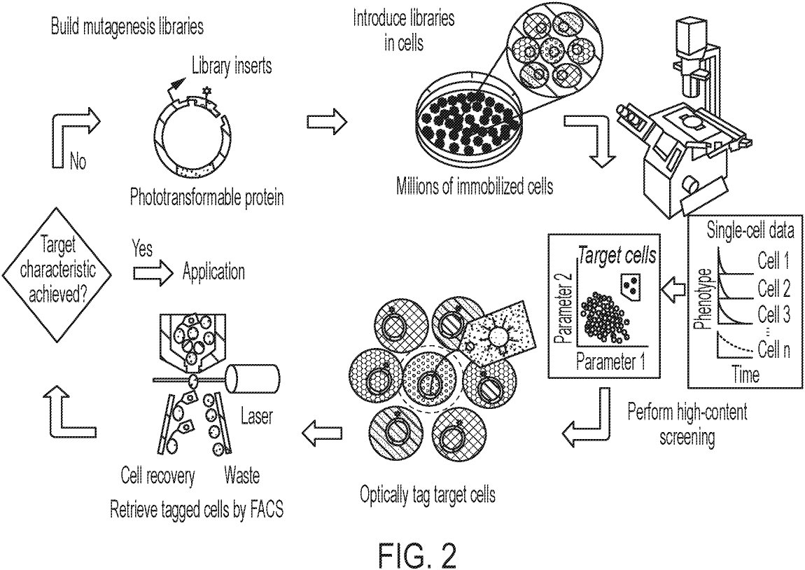

In order to rapidly evaluate thousands of possible variants, a high-throughput screening platform was applied. FIG. 2 illustrates screening process as applied to engineered mutagenesis libraries. In this embodiment, mutagenesis libraries are prepared with a vector coexpressing a phototransformable protein and introduced to host cells, such that individual cells only express a single variant. These cells are then imaged using a motorized microscope. The phenotypic characteristics of each individual cell are quantified by automated image analysis, and cells meeting certain target criteria are identified as target cells. These target cells are then optically marked for isolation by individually activating the phototransformable protein. The tagged target cells are detected and isolated from non-tagged cells using FACS. These sorted cells can be regrown for further characterization, to subject their plasmids to additional rounds of screening, or both.

Mutagenesis libraries were developed by simultaneously randomizing 3 predefined residues of the mVenus starting template. Overall, 8 mutagenesis libraries were built that targeted 21 different residues of mVenus. For each library, 8 amino acids were simultaneously mutated, resulting in 8,000 possible combinations. Degenerate codons (NNS) were used to randomize each position with all 20 amino acids during polymerase chain reaction (PCR). The overlap regions between inserts and linearized vector were designed as 18 bp in length. A cloning kit was used for seamless DNA cloning. To maximize the transformation efficiency, the cells were heat-shocked for 1 hour and co-transformed with high purity single stranded DNA (ssDNA). Transformed yeast were plated on uracil drop-out agar plates and incubated at 37° C.

72 hours post-transformation, the colonies on the transformation plates were collected using a cell scraper into 1 mL of sterile water. Up to 54,400 colonies were obtained per library, which corresponded to approximately 100,000 to 700,000 cells per library. The cells were washed 3 times with water and plated on glass bottom plates coated with a 0.1 mg/mL solution of poly-L-lysine. The attached cells were washed twice with water. After the final wash, water was replaced with PBS.

The yeast cells were immobilized on a glass bottom plate. Immobilized single cell libraries were imaged by widefield fluorescence imaging. Widefield imaging was performed using an inverted microscope equipped with a motorized XY stage with linear encoders, a hardware autofocus module, a 20X 0.75-NA objective, and imaging software. Excitation light was emitted from a solid-state multi-spectral light engine. Excitation and emission light were routed to and from the sample, respectively, using multi-band dichroic mirrors located in a filter cube below the objective turret. Emission light was filtered with multi-band filters. To separate blue and yellow fluorescence, the beamsplitter was fitted with a dichroic mirror. Blue fluorescence was further filtered by a 450/50 m emission filter. To separate green and red fluorescence, the beamsplitter was fitted with a separate dichroic mirror. Red fluorescence was further filtered by a 632/60 m emission filter.

Automated imaging was performed by sequentially scanning 64 to 169 nonoverlapping field-of-views. Because the illuminated area (-1.2 mm²) was larger than the field-of-view captured by the camera (-0.43 mm²), the fields-of-view were spaced to avoid imaging previously illuminated areas. Yellow and blue fluorescence images were acquired for each field-of-view at 0, 22.5, and 45 s. Yellow fluorescence was imaged using 508/25-nm excitation light at 20 mW/mm² and a 50 ms exposure time. To image blue fluorescence, a 395/25-nm light was used with lower irradiance (3.2 mW/mm²) to minimize non-selective photoactivation of PAmCherry1 during imaging. To ensure sufficient signal, a longer exposure time (400 ms) was used.

Photobleaching was performed by illuminating 508/25-nm excitation light at 20 mW/mm² for 45 s. Excitation light irradiance was calculated by dividing the measured power with the illumination area. For each objective used, the illumination area was determined by photoactivating a field-of-view of a dense culture of PAmCherry1-expressing yeast cells; only the photoactivated cells emit red fluorescence, and therefore, the region that show red fluorescence correspond to the illuminated area.

Images were segmented to identify individual cells. Supervised pixel classification was performed on a single representative image of the blue channel (reference TagBFP image) at the 0 s time point using machine learning-based segmentation software, which generated a binary segmentation mask. The generation of segmentation masks for all additional fields-of-view as accelerated using code that extracts segmentation parameters from the initial segmentation mask and conducts segmentation of several images in parallel. Depending on the total number of fields-of-view and the degree of parallelization, segmentation speeds of ~3 s per field-of-view or ~5 min for a typical library were achieved. Because cells were immobilized and exhibited minimal movement during imaging, the segmentation mask from the initial time point could be used for later time points. Channel registration between the blue (segmentation) and yellow (test) channels was performed before applying the segmentation mask to the yellow channel.

To analyze the individual cells identified above, the mean yellow and blue channel pixel intensities (F(Y) and F(B), respectively) of each cell was computed for each time point (t). The brightness (B) of each cell was defined as the ratio of its yellow and blue mean pixel intensities at t₀, the initial time point, i.e. B = F(Y, t₀) / F(B, t₀). Normalization by blue fluorescence was performed to reduce cell-to-cell variation caused by the variation of plasmid copy number. For screening experiments, the photostability (P) was defined as the fluorescence remaining after a period of continuous illumination, expressed as a fraction of the initial fluorescence, i.e. P = F(Y, tf) / F(Y, t₀), where t_{f} is the final time point.

To increase the chance of the selected variant cells being significantly superior to the parental cells, a probabilistic model was generated. After brightness and photostability values were quantified for all cells, the joint cumulative distribution function (JCDF) of these two scores of the parental cells was computed using kernel density estimation. Using the brightness and photostability values of each cell of a given library, the JCDF was used to compute the probability of the library cells being better than the parental using the JCDF. Depending on the number of promising candidates, 60 to 200 cells were selected for optical tagging and recovery. Cell selection was automated but could be refined after manual inspection if desired.

The locations of the target cells were determined from the images metadata and used to automatically position the microscope stage so that target cells are located in the center of the field-of-view. Using a digital mirror device (DMD), photoactivation was performed on a 4 µm-by-4 µm region centered on the target cells. The photoactivation light irradiance measured at the sample plane was 88 mW/mm². Photoactivation occurred for 45-60 s. The microscope automatically and sequentially photoactivated target cells. For each target cell, red fluorescence images were taken using 550/15 nm light at 67 mW/mm² before and after photoactivation, and the emitted light was filtered by a 632/60-nm filter.

After photoactivation, all cells were detached from the glass-bottom plates by removing the culture medium and incubating the cells in a trypsin-EDTA solution for 10 min at 37° C. Cells were pipetted up and down rigorously to promote detachment and resuspension into single cells. Detached cells were washed once and resuspended with phosphate-buffered saline (PBS).

A flow cytometry cell sorter was used to detect and retrieve cells that were optically tagged. FlowJo software was used to analyze flow cytometry data. Two controls were used to determine gates for photoactivated cells, both expressing the parental mVenus, PAmCherry1, and the reference TagBFP. The negative control (with no photoactivation) had no photoactivated cells while the positive control (1 field-of-view photoactivation) corresponds to photoactivation of several hundreds of cells in a single field-of-view. The two controls were compared and the photoactivated cell gate was determined by selecting the region that had cells present only in the positive control.

Using the flow cytometry cell sorter, photoactivated cells were sorted in a 5-mL tube containing synthetic drop-out media and plated on a synthetic drop-out agar plate. All photoactivated cells were sorted into a single tube, plated onto an agar plate, and incubated at 37° C. for 72 hours to enable colony formation.

Colonies grown from the sorted sample were grown in synthetic drop-out media for population-level analysis. Plasmid DNA was prepared from promising variants using a yeast plasmid miniprep kit and sequenced to identify novel mutations.

Eight rounds of screening identified four variants that exhibited longer photobleaching half-lives in yeast and mammalian cells without any loss in brightness as compared to the original mVenus precursor.

### Example 2. In Cellulo Characterization of Fluorescent proteins

### Preparation of Yeast Cells

Yeast cells expressing fluorescent proteins were streaked on synthetic drop-out agar plates from glycerol stocks. Individual colonies were picked and grown overnight at 37° C. in a synthetic dropout media buffered at pH 7.0 with 10 mM HEPES. The growth temperature and pH were not standard for yeast (which are typically grown at 30° C. and at acidic pH between 4 and 6) but were chosen so that the results would be most relevant to expression in mammalian systems which are normally cultured at 37° C. and pH 7.3. A pH 7.0 was used instead of 7.3 because a pH higher than 7.0 severely stunt growth of yeast cells. For each variant, three colonies were selected and grown in separate culture tubes overnight until saturation. Cells were diluted ~20 fold, regrown to mid-log phase which corresponded to OD600 = 0.5 when measured with a spectrophotometer. Cells were washed 3 times with sterile water, and immobilized on poly-L-lysine-coated microplate for 15 minutes. Water was replaced with PBS for imaging.

### Preparation of Mammalian Cells

FP expression plasmids were transfected in HEK293A cells using FuGENE HD Transfection Reagent (Promega) following the manufacturer’s instructions, except that cells were transfected with 200 ng DNA and 0.6 µL FuGENE per well of a 24-well plate, or 100 ng of DNA and 0.3 µL of FuGENE per well of a 96-well plate. Transfected cells were placed on a poly-L-lysine-coated microplate and incubated at 37° C. in air with 5% CO₂ for 2 days. Just before imaging, cells were washed once with Dulbecco’s PBS (DPBS), and the growth media was replaced with Hank’s Balanced Salt Solution (HBSS) supplemented with 10 mM HEPES.

### Photobleaching Under Widefield Illumination

Widefield imaging was performed using an inverted microscope equipped with a motorized XY stage with linear encoders, a hardware autofocus module, a 20X 0.75-NA objective, and imaging software. Excitation light was emitted from a solid-state multi-spectral light engine. Excitation and emission light were routed to and from the sample, respectively, using multi-band dichroic mirrors located in a filter cube below the objective turret. Emission light was filtered with multi-band filters. To separate blue and yellow fluorescence, the beamsplitter was fitted with a dichroic mirror. Blue fluorescence was further filtered by a 450/50 m emission filter. To separate green and red fluorescence, the beamsplitter was fitted with a separate dichroic mirror. Red fluorescence was further filtered by a 632/60 m emission filter.

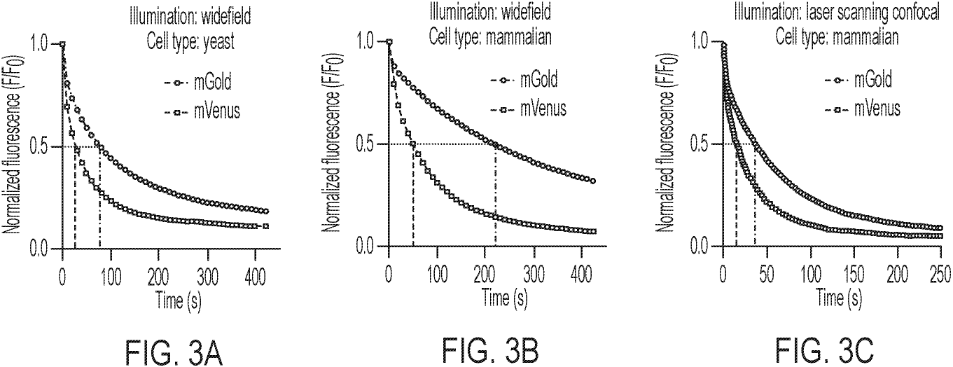

FIG. 3A shows a chart comparing the photostability of mGold and wild-type mVenus in yeast cells that were photobleached with widefield illumination. Yeast cells were transformed with plasmids constitutively expressing mGold or mVenus and attached to a glass bottom plate. Several thousand cells were photobleached using continuous widefield 508/25 nm illumination at 20 mW/mm 2 for 420 s. Yellow fluorescence images were taken every 10 s during photobleaching. The fluorescence intensity was normalized to the initial fluorescence (F/F₀). Photobleached cells were analyzed and the mean photobleaching behaviour was determined by calculating the mean normalized fluorescence of the analyzed cells at each time point. The experiment was prepared in triplicate by picking and assaying cells from 3 different colonies. Mean values of triplicates are shown. The shaded areas represent the s.e.m. of the triplicates. The area under the curve for each sample was computed and the unpaired two-tailed t test was conducted (p ≤ 0.0001). In yeast, mGold has significantly greater photostability than mVenus using widefield illumination.

FIG. 3B shows a chart comparing the photostability of mGold and wild-type mVenus in mammalian cells that were photobleached with widefield illumination. Mammalian cells (HEK239A) were transiently transfected with plasmids constitutively expressing mGold or mVenus and attached to a glass bottom plate. Several hundred cells were photobleached. The same photobleaching setup and analysis described in FIG. 3A were used. The experiment was prepared in triplicate by assaying cells from 3 independent transfections. Mean values of triplicates are shown. The shaded areas represent the s.e.m. of the triplicates. The area under the curve for each sample was computed and unpaired two-tailed test was conducted (p ≤ 0.0001). Based on these results, mGold has significantly greater photostability the mVenus in mammalian cells using widefield illumination.

### Photobleaching Under Laser-Scanning Microscopy

FIG. 3C shows a chart comparing the photostability of mGold and wild-type mVenus in mammalian cells that were photobleached with laser scanning confocal illumination. Mammalian cells (HEK239A) cells were prepared as described in FIG. 3B. Cells expressing mGold or mVenus were continuously photobleached and imaged using a laser scanning confocal microscope. A 514 nm laser was used at 5% power corresponding to 32 µW. The mean photobleaching curves are shown. The shaded areas represent the s.e.m. n = 17 (mVenus) or n = 15 (mGold) cells. The area under the curve for each sample was computed and the unpaired two-tailed t-test was conducted. p ≤ 0.0001. As shown with widefield illumination, mGold still maintains higher photostability (~2-fold higher) than mVenus in mammalian cells photobleached by laser scanning confocal illumination.

### Image Analysis of Brightness and Photostability Data

For each photostable fluorescent protein variant, population-level analysis was conducted. The fields-of-view were segmented to compute single-cell brightness and photostability scores. All the segmented cells in the 2 to 3 fields-of-view were combined, leading to a total of ~500-8,000 yeast cells and ~100-200 mammalian cells. The brightness and photostability of individual cells were computed after background subtraction and outliers were removed using the ROUT (Q=1%) method. Mean brightness and photostability values were determined to obtain a population-level performance of the variants.

To analyze the brightness of individual cells, the mean yellow and blue channel pixel intensities (F(Y) and F(B), respectively) of each cell was computed for each time point (t). The brightness (B) of each cell was defined as the ratio of its yellow and blue mean pixel intensities at t₀, the initial time point, i.e. B = (Y, t₀)/(B, t₀). Normalization by blue fluorescence was performed to reduce cell-to-cell variation caused by the variation of plasmid copy number. For screening experiments, the photostability (P) was defined as the fluorescence remaining after a period of continuous illumination, expressed as a fraction of the initial fluorescence, i.e. P = ((Y, tf) / (Y, t₀), where t_{f} is the final time point.

. The photobleaching half-life, the time required for the fluorescence to decrease by half, was calculated as a standard metric for photostability. For each variant, yeast cultures were grown from three independent colonies, and three independent transfections were conducted in mammalian cells. The mean and standard deviation of the brightness and photostability from these triplicates were calculated and compared for statistical analysis.

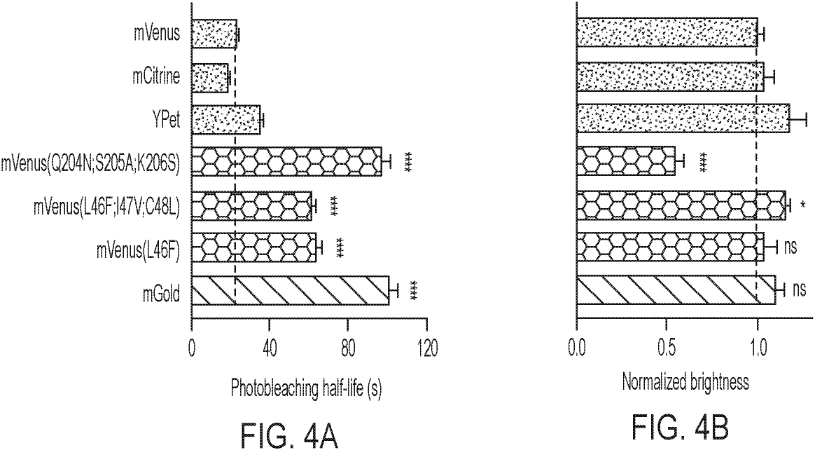

FIG. 4 shows charts comparing, in yeast, the photostability (FIG. 4A) and brightness (FIG. 4B) of four photostable yellow fluorescent protein variants (mGold, mVenus(L46F), mVenus(L46F; I47V; C48L), and mVenus (Q204N; S205A; K206S)) to three commonly used yellow fluorescent proteins (mVenus, mCitrine, and YPet). The yeast expression vector also co-expressed TagBFP to normalize for variation in copy number and expression capacity. For each variant, 2,000-7,000 cells were analyzed to determine the mean photobleaching half-life (left) and the mean relative brightness (the ratio of yellow fluorescence and blue fluorescence) normalized to that of mVenus(right). Samples were prepared in triplicates and the bars represent the mean values of the triplicates. Error bars indicate the s.e.m. of the triplicates. p ≤ 0.0001 for both brightness and photostability (one-way ANOVAs). The p values of post hoc comparisons with mVenus used the Bonferroni correction for multiple comparisons and are shown on the plot: ‘ns’, not significant; *, p ≤ 0.05; ***, p ≤ 0.0001. FIG. 4A shows that all four variants exhibit significantly improved photostability compared to any of the common YFPs. The variants exhibiting the least improved photostability, mVenus(L46F) and mVenus(L46F; I47V; and C48L) still exhibited approximately 2.7-fold larger photobleach half-life. FIG. 4B illustrates the fact that three of the variants have a similar brightness to the common YFPs. The fourth variant, mVenus (Q204N; S205A; K206S), exhibit approximately half the brightness of the other YFPs including the other variants.

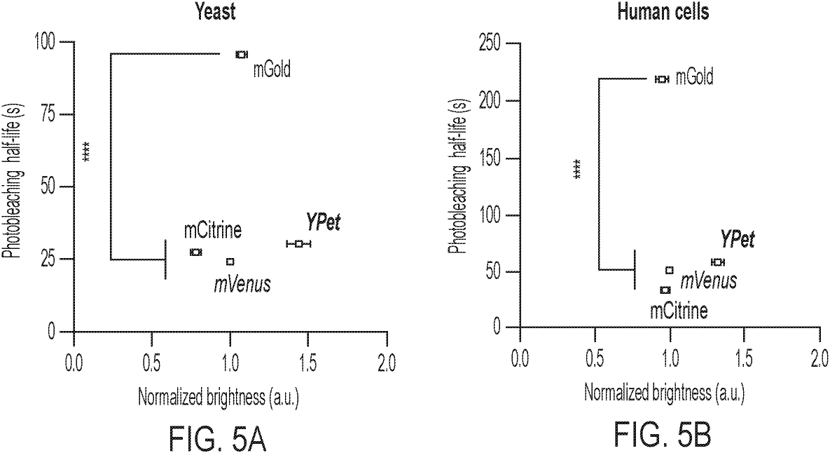

FIG. 5 shows charts comparing photostability and brightness of mGold with mCitrine, Ypet, and wild-type mVenus in yeast (FIG. 5A) and human cells (FIG. 5B). Cells were photobleached with 508/25-nm light at 20 mW/mm². YFP brightness was normalized for cell-to-cell differences in protein expression (YFP/BFP). The square markers indicate the means from six yeast cultures or six independent transfections. For each replicate, the mean photobleaching half-life and brightness of several hundred yeast or human cells were determined. The error bars represent the SEM. P ≤ 0.0001 for Welch’s analysis of variance (ANOVA) for both yeast and human cell data: ****P ≤ 0.0001 for Dunnett’s T3 post hoc test. mGold was more photostable than commonly used YFPs and exhibited similar brightness in both yeast and human cells.

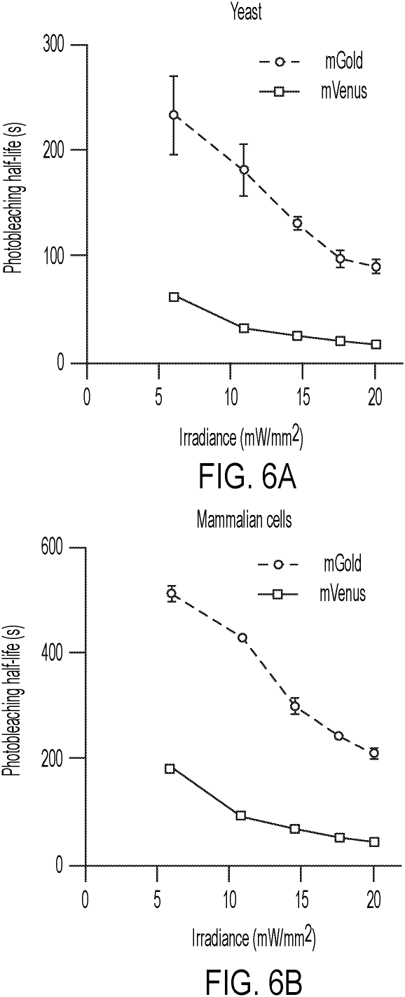

FIG. 6 shows charts of the photostability at different irradiances in yeast (FIG. 6A) and mammalian cells (FIG. 6B). Even while increasing the intensity of the illumination, mGold maintains greater photostability than wild-type mVenus.

### Imaging Mgold Fusion Constructs

Confocal imaging was conducted to evaluate whether mGold fused to different organelle/cytoskeleton-localization tags/proteins would produce fluorescence with the expected pattern of subcellular localization. HeLa cells were transfected with FuGENE as described above. Cells were placed on glass bottom plates without poly-L-lysine coating because poly-L-lysine promoted detachment with HeLa cells, producing rounder cells after the PBS wash step. Transfected HeLa cells were washed with PBS and resuspended in HBSS supplemented with 10 mM HEPES. Laser-scanning confocal images were obtained using a high-speed confocal microscope driven by the Zen software (version 2.3 SP1). Images were acquired with a 40X-1.1 NA water immersion objective, a 488-nm argon laser at 3% power, and a per-pixel dwell time of 2 µs. Emission light was filtered using a multipass beamsplitter and acquired with a 32 channel GaAsP detector with a detector gain of 740, and 1-Airy unit pinhole size. To increase the signal-to-noise ratio, 2 scans were performed and averaged for each image.

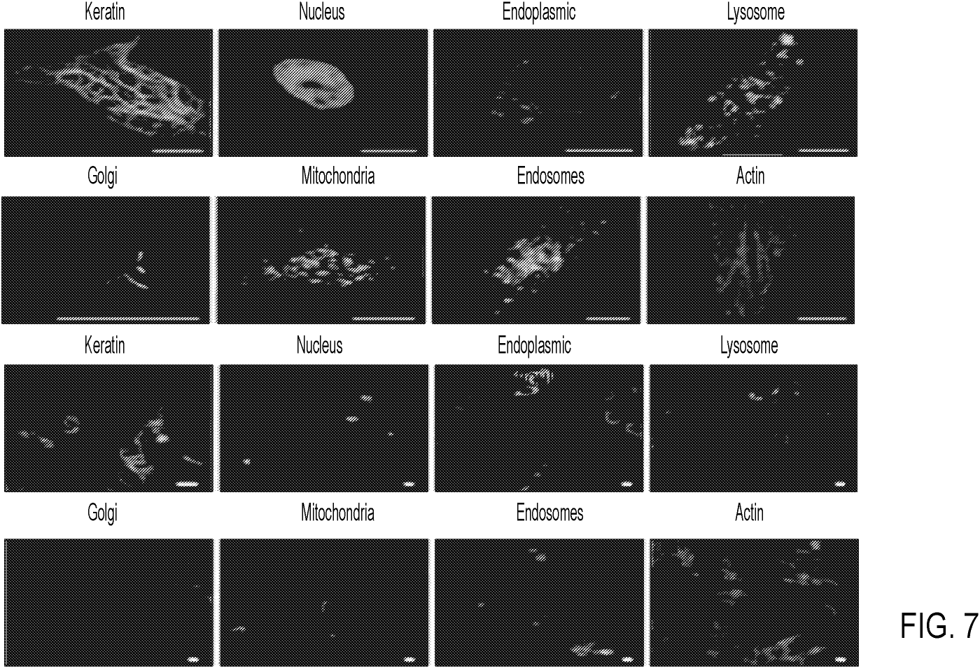

FIG. 7 shows images of mGold subcellular localization in HeLa cells. Indicated in each construct is the name of mGold’s fusion partner, the position of the fusion (N- or C-terminal with respect to the fluorescent protein), and the number of residues in the linker between the two proteins. Specifically, FIGS. 7A-7H show mGold-KRT18-N-17 (Cytoskeleton, keratin); mGoldChafla-C-10 (Nucleus); mGold-CANX-N-14 (Endoplasmic reticulum); mGold-Lamp1-N-20 (Lysosome); mGold-Man2a1-N-10 (Golgi apparatus); mGold-COX8A-N-8 (Mitochondria); mGold-RAB4A-C-7 (Endosome); and mGold-ACTB-C-18 (Cytoskeleton, actin), respectively. FIGS. 7I-7P show zoomed out images of the cells expressing the mGold constructs. Upon appending mGold to 8 proteins or peptides with different subcellular locations, mGold produced the expected pattern of fluorescence in all cases, suggesting its suitability as a fusion partner.

### Comparing Cytotoxicity

To evaluate cytotoxicity, the assay was conducted according to a published methodology (Shemiakina et al., Nat. Commun. (2012)). Fluorescent protein expression plasmids expressing EGFP, mGold, or mVenus were transfected separately in HeLa cells. For each fluorescent protein plasmid, cells were transfected following the manufacturer’s instructions and used 2 µg of DNA and 6 µl FuGENE. Transfected cells were placed in a well of a 6-well microplate and incubated at 37° C. in air with 5% CO₂. Two days after transfection, cells were detached by incubating the cells in a trypsin-EDTA solution for ~5 min. EGFP cells were mixed with mGold or mVenus cells to produce two mixed populations: (i) EGFP⁺ and mGold⁺ mixed cells and (ii) EGFP⁺ and mVenus⁺ mixed cells. The proportion of GFP⁺ and YFP⁺ cells in each mixed cell population was analyzed using an Attune NxT flow cytometer. Mixed population of cells were diluted 10-fold and plated into three wells of a six-well plate. Five days after transfection, mixed cells were detached and the proportion of GFP⁺ and YFP⁺ cells was analyzed using flow cytometry. For both flow cytometry analyses, single-fluorescent protein controls (cells expressing only EGFP, mVenus, or mGold) and negative control (cells that were not transfected) were prepared to compensate for the spectral overlap between EGFP and YFPs and to design analysis gates for GFP⁺ and YFP⁺ cells. Only live cells were analyzed by identifying and removing dead cells from analysis. NucBlue DAPI (4′,6-diamidino-2phenylindole) stain was used to stain dead cells. Cytotoxicity was calculated using the flow cytometry data from both day 2 and day 5 using the following formula: ((% of YFP⁺ cells at day 2) / (% of YFP⁺ cells at day 5))/((% of EGFP⁺ cells at day 2) / (% of EGFP⁺ cells at day 5)).

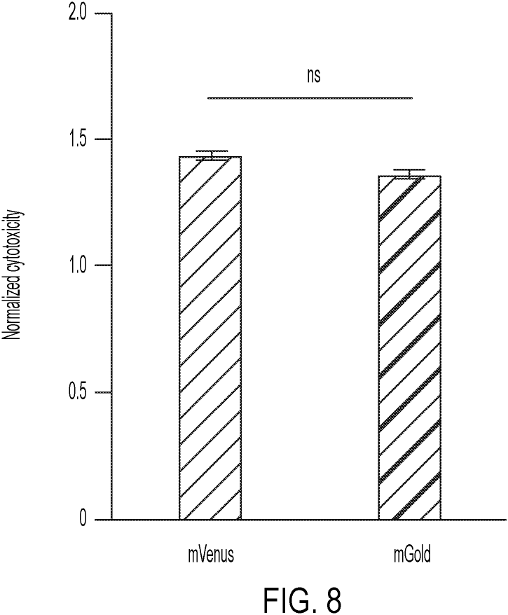

FIG. 8 shows the cytotoxicity profiles of mGold and mVenus. The mean values of n = 3 independent transfections are shown. The error bars indicate SEM. The Mann-Whitney U test was used for statistical analysis: ‘ns’ = not significant.

### Example 3. In Vitro Characterization of Fluorescent Proteins

In vitro characterization of fluorescent proteins was conducted by adapting published methods (Ai, H.-W., et. al, Nat. Protoc. (2014)).

### Expressing and Purifying Fluorescent Proteins

Bacterial cells from E.coli strain BL21 were grown to an OD₆₀₀ of 0.1 and induced using 1 mM isopropyl β-d-1-thiogalactopyranoside (IPTG) for 8 hours. FPs were purified using the Ni-NTA Fast Start Kit (Qiagen) and dialyzed in 50 mM or 5 mM tris(hydroxymethyl)aminomethane buffer (tris) at pH 7.5. Protein concentrations were determined using the Pierce BCA Protein Assay Kit.

### Obtaining the Excitation and Emission Spectra

FPs were diluted in 50 mM tris buffer at varying concentrations. A plate reader was used to produce excitation and emission spectra. Excitation scan data utilized excitation wavelengths from 400 to 555 nm and collected emission intensity at 580/10 nm. Emission scan data used an excitation wavelength of 475/10 nm and utilized emission wavelengths from 500 to 700 nm. Data was also collected for a control well with only 50 mM Tris buffer and no fluorescent proteins. Excitation and emission curves were created by subtracting the blanks from each sample, normalizing each individual well to have a maximum fluorescent intensity value of 1, then averaging the curves for each FP. The excitation and emission peaks were determined from these averaged curves.

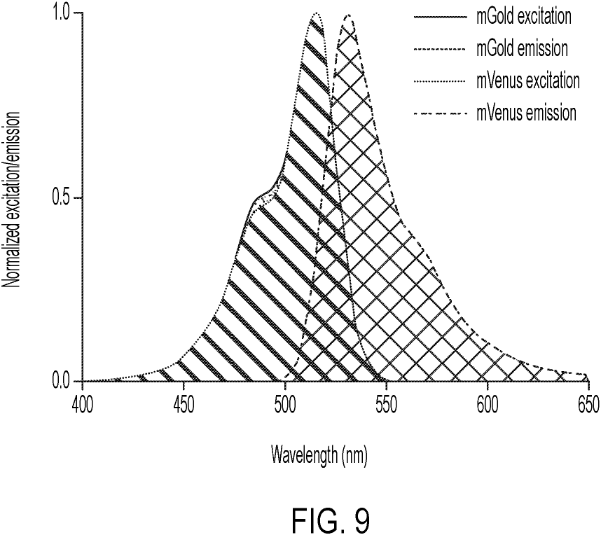

FIG. 9 shows the in vitro excitation and emission spectra of mGold as compared to wild-type mVenus. The mGold spectra is overlayed with the excitation (dashed line) and emission (dotted line) spectra of mVenus. The excitation and emission spectra of mGold are similar to those of mVenus.

### Quantifying the Extinction Coefficient and Quantum Yield

A spectrometer was first used to measure the absorbance at 475 nm of purified FPs and of a reference dye, rhodamine 123. Based on these values, each sample was diluted to target absorbances of 0.0035, 0.0028, and 0.0021 on a 96 well plate as to eliminate the inner filter effect. Rhodamine 123, a dye with excitation/emission maxima of 507 and 529 nm respectively, was diluted in ethanol, while FPs were diluted in 5 mM Tris buffer. Undiluted samples of both rhodamine 123 and the FP samples were plated. A plate reader was then used to quantify fluorescence using excitation at 475 nm and emission at 580 nm. The fluorescent intensities of the diluted and stock samples were compared to calculate the real absorbance of the dilutions using the formula A_{diluted} = A_{stock} *(F_{diluted} / F_{stock}). The slope of each sample’s emission vs. absorbance curves were determined using linear regression (intercept = 0). Quantum yields (QY) were calculated using the formula QY_{FP} = QY_{St} × (S_{St} / S_{FP}) × (R_{FP} / R_{St}), where S is the slope of the sample’s curve and R is the refractive index of the solvent used. The FP subscript refers to the fluorescent protein sample, while St refers to the rhodamine 123 standard. Extinction coefficients were determined with a spectrometer by finding peak absorption, the wavelength of which was determined earlier using an absorbance sweep reading with the plate reader. Three dilutions of each FP variant in 50 mM tris were measured with the spectrometer in 1-cm quartz cuvettes. The extinction coefficient (ε) was determined using the Beer-Lambert law (A = ε*1*C or ε = A/(1*C); where A is absorbance, 1 is path length in cm, and C is molar concentration) after correcting for the dilution of the sample. ε was calculated by averaging the values obtained at different dilutions.

### Quantifying the PKA

To determine pKa, a series of buffers were prepared with pH in the range of 3-10. Buffers with pH 3-5.5 were made with 100 µM citric acid 100 µM Na citrate, buffers with pH 6-8 were made with 100 µM KH₂PO₄ and 100 µM NaH₂PO₄, and buffers with pH 8.5-12 were made with 100 µM glycine and 100 µM NaOH. All buffers were then adjusted to the desired pH using HCl or NaOH. 100 µL of each pH buffer were loaded into a 96-well glass-bottom imaging plate with 10 µL of 1 µM protein samples dialyzed in 5 mM Tris in four replicates. A plate reader was then used to determine emission intensity of each FP at 530 nm using 500 nm excitation light. Emission intensity vs. pH was then normalized to the intensity value at pH = 10 and plotted. Linear interpolation was used to determine the pKa, defined as the pH at which fluorescence is 50% of its maximum.

### Quantifying Photostability

A photobleaching chamber was constructed using a glass microscope slide as the base, then mounting a coverslip on top of two additional spacer coverslips attached to the base with tape. A 1 µM dilution of each FP was mixed with 20% wt/vol acrylamide/bis-acrylamide, 30% wt/vol solution, 3 µL of 10% ammonium persulfate, and 0.5 µL of TEMED in a 1.5 mL tube. Right after adding TEMED, ~80 µL of the solution was transferred to the space between the top coverslip and glass slide. The gel was allowed to polymerize for ~5 min. The edges were sealed with Cytoseal 60. After 12 hours, the slides were continuously photobleached for 7 minutes with 5 s interval image captures using widefield 510/25 nm light with 62.5 mW/mm² (using 60X oil objective). For each FP, 9 fields-of-view were tested. For each field-of-view, the photobleaching half-life was calculated by determining the time which the fluorescence reached half of initial fluorescence. The mean photobleaching half-life values were calculated and plotted.

TABLE 1

In Vitro Characterization of Photophysical Properties of Mgold

| Protein | λₑₓ^{a} | λₑₘ^{b} | ε^{c} | Ψ^{d} | Molecular Brightness^{e} | pKa^{f} | Photobleaching Half-Life (s)^{g} |
|---|---|---|---|---|---|---|---|
| mGold | 515 | 531 | 107 ± 6 | 0.64 | 68 | 5.8 | 29.8 ± 0.3 |
| mVenus | 515 | 532 | 110 ± 6 | 0.65 | 72 | 5.7 | 10.1 ± 0.4 |

^{a} Excitation maximum (in nm).
^{b} Emission maximum (in nm).
^{c} Quantum yield of fluorescence.
^{d} Extinction coefficient, in mM -1 cm-1.
^{e} Calculated as product of ε and Ψ.
^{f} Calculated as the pH at which the in vitro fluorescence intensity is half of its maximal value (standard error of mean for n = 3 measurements was < 0.1).
^{g} Time taken for fluorescence intensity to reach half of its initial value under 510/25 nm light with 62.5 mW/mm2 using widefield 510/25 nm as the light source. Samples were subjected to continuous illumination during this time.

### Determining Oligomeric State

Size exclusion chromatography demonstrates that mGold is a monomer at 10 µM in vitro. tdTomato (tandem dimer), mCherry (monomer), and mVenus (monomer) were used as size standards. Each fluorescent protein was at 10 µM (6.5 µM for tdTomato) and was run separately. mVenus and mGold were detected by measuring the absorbance at 515 nm. tdTomato and mCherry were detected by measuring the absorbance at 555 nm and 587 nm, respectively. Absorbance values were normalized to the maximum absorbance.

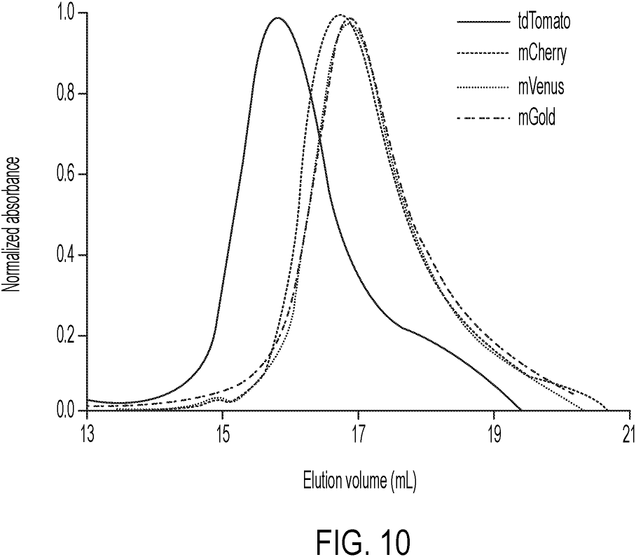

FIG. 10 shows the elution profile of mGold (dashed line) as compared to wild-type mVenus (dotted line), mCherry (solid line), and tdTomato (dashed-dotted line). The elution profile of mGold is similar to that of mVenus and mCherry monomers.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A photostable fluorescent protein variant comprising an amino acid sequence that is at least 90% identical to SEQ ID NO:3, wherein the amino acid sequence comprises a first substitution at a residue corresponding to residue 46 of a polypeptide of SEQ ID NO:2, and the variant is more photostable than the polypeptide of SEQ ID NO:2.

2. The photostable fluorescent protein variant of claim 1, wherein the first substitution is an L46F substitution.

3. The photostable fluorescent protein variant of claim 1, wherein the amino acid sequence further comprises a second substitution at a residue corresponding to residue 63 of the polypeptide of SEQ ID NO:2.

4. The photostable fluorescent protein variant of claim 3, where the first substitution is an L46F substitution and the second substitution is a T63S substitution.

5. The photostable fluorescent protein variant of claim 3, wherein the amino acid sequence further comprises a third substitution at a residue corresponding to residue 163 of the polypeptide of SEQ ID NO:2.

6. The photostable fluorescent protein variant of claim 5, where the first substitution is an L46F substitution, the second substitution is a T63S substitution, and the third substitution is an A163V substitution.

7. The photostable fluorescent protein variant of claim 3, wherein the amino acid sequence further comprises a third substitution at a residue corresponding to residue 78 of the polypeptide of SEQ ID NO:2.

8. The photostable fluorescent protein variant of claim 7, where the first substitution is an L46F substitution, the second substitution is a T63S substitution, and the third substitution is an M78L substitution.

9. The photostable fluorescent protein variant of claim 3, wherein the amino acid sequence further comprises a third substitution at a residue corresponding to residue 151 of the polypeptide of SEQ ID NO:2.

10. The photostable fluorescent protein variant of claim 9, where the first substitution is an L46F substitution, the second substitution is a T63S substitution, and the third substitution is an Y151C substitution.

11. The photostable fluorescent protein variant of claim 3, wherein the amino acid sequence further comprises a third substitution at a residue corresponding to residue 147 of the polypeptide of SEQ ID NO:2 and a fourth substitution at a residue corresponding to residue 156 of the polypeptide of SEQ ID NO:2.

12. The photostable fluorescent protein variant of claim 11, where the first substitution is an L46F substitution, the second substitution is a T63S substitution, the third substitution is an S147N substitution, and the fourth substitution is a K156R substitution.

13. The photostable fluorescent protein variant of claim 3, wherein the amino acid sequence further comprises a third substitution at a residue corresponding to residue 80 of the polypeptide of SEQ ID NO:2, a fourth substitution at a residue corresponding to residue 147 of the polypeptide of SEQ ID NO:2, and a fifth substitution at a residue corresponding to residue 232 of the polypeptide of SEQ ID NO:2.

14. The photostable fluorescent protein variant of claim 13, where the first substitution is an L46F substitution, the second substitution is a T63S substitution, the third substitution is an Q80R substitution, the fourth substitution is a S147C substitution, and the fifth substitution is a G232S substitution.

15. The photostable fluorescent protein variant of claim 1 ,wherein the amino acid sequence is at least 95%, 98%, or 99% identical to SEQ ID NO:3.

16. (canceled)

17. (canceled)

18. The photostable fluorescent protein variant of claim 1, wherein the amino acid sequence is identical to SEQ ID NO: 1, SEQ ID NO:3, SEQ ID NO:4, SEQ ID NO:6, SEQ ID NO:7, SEQ ID NO:8, SEQ ID NO:9, or SEQ ID NO: 10.

19. (canceled)

20. (canceled)

21. (canceled)

22. (canceled)

23. (canceled)

24. (canceled)

25. (canceled)

26. A photostable fluorescent protein variant comprising an amino acid sequence that is at least 90% identical to SEQ ID NO:5, wherein the amino acid sequence comprises a first substitution at a residue corresponding to residue 204 of a polypeptide of SEQ ID NO:2, a second substitution at a residue corresponding to residue 205 of the polypeptide of SEQ ID NO:2, and a third substitution at a residue corresponding to residue 206 of the polypeptide of SEQ ID NO:2, and the variant is more photostable than the polypeptide of SEQ ID NO:2.

27. The photostable fluorescent protein variant of claim 26, wherein the first substitution is a Q204N substitution, the second substitution is an S205A substitution, and the third substitution is a K206S substitution.

28. The photostable fluorescent protein variant of claim 26, wherein the amino acid sequence is at least 95%, 98%, or 99% identical to SEQ ID NO:5 or is identical to SEQ ID NO:5.

29. (canceled)

30. (canceled)

31. (canceled)

32. A fusion protein comprising the photostable fluorescent protein variant of claim 1 operatively linked to a protein of interest.

33. A tandem fluorescent protein comprising a first photostable fluorescent protein variant of claim 1 operatively linked to a second fluorescent protein.

34. A nucleic acid molecule, comprising a nucleic acid sequence encoding a photostable fluorescent protein variant of claim 1.

35. (canceled)

36. (canceled)

37. (canceled)

38. The nucleic acid molecule of claim 34, wherein the nucleic acid sequence encodes a fusion protein comprising the photostable fluorescent protein variant.

39. The nucleic acid molecule of claim 34, wherein the nucleic acid sequence is identical to SEQ ID NO:11, SEQ ID NO:12, SEQ ID NO: 13, SEQ ID NO:14, or SEQ ID NO:15.

40. (canceled)

41. (canceled)

42. (canceled)

43. (canceled)

44. A vector comprising the nucleic acid molecule of claim 34.

45. An expression cassette comprising:
a transcriptional initiation region that is functional in an expression host;
the nucleic acid molecule of claim 34 ; and
a transcriptional termination region function in the expression host.

46. A host cell, or progeny thereof, comprising the expression cassette according to claim 45 as part of an extrachromosomal element or integrated into the genome of a host cell as a result of introduction of the expression cassette into the host cell.

47. A transgenic cell, or progeny thereof, comprising the nucleic acid molecule of claim 34 .

48. A method of detecting a protein of interest, comprising the steps of:
a) expressing the fusion protein of claim 32; and
b) detecting the fluorescence of the fusion protein.

49. A method of detecting the subcellular localization of a protein of interest, comprising the steps of:
a) expressing in a cell the fusion protein of claim 32;
b) detecting the fluorescence of the fusion protein; and
c) determining the subcellular location of the fluorescence within the cell.

50. A method of detecting the motility of a protein of interest, comprising the steps of:
a) expressing in a cell the fusion protein of claim 32;
b) performing time-sequential observations of fluorescence in the cell; and
c) detecting differences in the fluorescence between the time-sequential observations.
